(19) United States
(12) Patent Application Publication
(10) Pub. No.: US 2026/0331238 A1
(43) Pub. Date: Oct. 22, 2026

(54) HEALTH ESTATE INSIGHTS AND RELATIONSHIP (HEIR) SYSTEM

(71) Applicant: Jason Robert Hillebrecht, Flemington, NJ (US)

(72) Inventor: Jason Robert Hillebrecht, Flemington, NJ (US)

(21) Appl. No.: 19/182,705

(22) Filed: Apr. 18, 2025

Related U.S. Application Data
(63) Continuation of application No. 19/182,702, filed on Apr. 18, 2025.

(51) Int. Cl. G16H 10/60; G06N 3/0455; G16H 50/30; G16H 50/70

(52) U.S. Cl. CPC G16H 10/60; G06N 3/0455; G16H 50/30; G16H 50/70

(57) ABSTRACT

An accurate and comprehensive family health history is a valuable tool for informing disease prevention, personalized medicine and family planning strategies. Unfortunately, the information clinicians receive from patients are often vague and non-actionable. The problem is exacerbated by health data privacy laws that make it difficult for family members to access health data for their relatives, both alive and deceased. The Health Estate Insights and Relationship (HEIR) System includes technology and methods for: managing multiple generations of family health data and the legal permissions needed to access and share this information, identifying and enriching gaps in family health data, and for generating family-level biologic signatures. The benefits of consolidating family-level biologic data include improved disease risk stratification, identification of familial clusters of diseases, improved targeting of personalized medicines and informed decision-making for family planning and fertility treatments, to name a few. System-related insights are shared at the point of care and with other authorized individuals and organizations through curated reports that do not compromise the privacy and/or identity of any contributing family member.

### TECHNICAL FIELD

The field of this invention is a system for family members or organizations to access, manage and/or derive actionable insights from a family health history that is accurate, comprehensive and persistent. The invention provides for the solicitation, storage and transfer of the legal permissions required to manage family health data across multiple generations of family members. The invention enables family members to connect with, and verify the authenticity of, other family members and/or representatives with the legal authority to manage the personal health information (PHI) of a family member. Users of the current invention can selectively load patient and/or external data into the system and choose the type and scope of health data they wish to share with one or many verified family members.

The invention includes features for intelligently identifying and enriching gaps in an individual's family health history and for deriving actionable insights through methods in advanced analytics and data science. Insights include but are not limited to the generation of a family-level biologic signature that consolidates and organizes biologic markers (e.g., genes, metabolites, proteins) for a group of family members. The insights derived from the current invention are summarized and shared with authorized parties, in ways that do not compromise the identity and/or privacy of the contributors of family health data. The insights derived and provided by the current invention inform decisions relating but not limited to: disease prevention, diagnostic screening, family and fertility planning, clinical trial opportunities, health risk scoring, legal, financial and family estate planning.

### BACKGROUND

A commonly overlooked aspect of traditional estate planning is the gathering of the permissions required to access the medical records of a family member. While it is not uncommon for individuals who are aging or unwell to name a healthcare proxy to assist in making medical decisions, it is far less common for family members to proactively gather the consents required to access a relative's health data, in the event of their death. A major challenge to accessing the health records of departed family members is that the Health Insurance Portability and Accountability Act's (HIPAA) Privacy Rule protects the individually identifiable health information about a decedent for 50 years following the date of death of the individual. With nearly twenty percent of the U.S. population aging 65 years and older, there is a growing need to expand the scope of traditional estate planning so that the health data of aging or unwell family members can be accessed by their relatives.

Patients traditionally derive their family health history through anecdotal and secondhand reports from relatives. These collection methods often yield a vague or incomplete picture of an individual's family health history. Taken together with low health literacy rates and the social stigma associated with sharing medical information amongst family members, many individuals miss out on potentially lifesaving screenings and disease prevention strategies.

Consolidating the permissions needed to access the health data for a group of family members is a critical requirement for creating an accurate family health history. The second challenge to creating a comprehensive family health history is that the data often reside in multiple, unrelated healthcare data management systems. While modern patient records are collected and maintained through digital systems, older medical charts may exist outside of any database. These data are almost never linked and often reside in different healthcare organizations, practice-facilities and their disparate management systems. There is currently no system that is designed to gather, store and interpret medical, biologic and genetic data for a verified group of family members.

Family health histories, based on actual medical and healthcare claims data, are derived by health insurance plans for individuals covered by a common insurance policy (e.g., policy subscribers, spouses, dependents) or by healthcare providers and patients who have access to the medical records of their children under the age of 18, with some exceptions. In both cases, patients rarely have direct access to, or ownership of, a comprehensive view of their family health history data. Outside of parent-child relationships, legal guardians and healthcare proxies, there is very little sharing of medical data across family members, especially when they are covered by different healthcare insurance companies and/or treated by different healthcare provider organizations.

There is also no easy way for an individual or organization to compare a comprehensive family health history to: one or many personalized medicines, data registries of rare and inheritable diseases, a fertility donor's biologic data, and clinical trial inclusion criteria to name a few.

Taken together, there is a meaningful opportunity to combine methods in advanced analytics and data science, with family health and reference data to deliver valuable insights to the point of care and for family, legal, life and estate planning.

### Summary of the Embodiments

The present disclosure relates to an advanced system for optimizing and expanding existing methods of consolidating, managing, and sharing family health data and derived insights with authorized individuals, parties, agencies, institutions, and organizations. These include, but are not limited to, family members, legal guardians, government or private agencies with jurisdiction over family members or their data, healthcare providers, adoption agencies or designated adoption services, adoptive parents or guardians, diagnostic and genetic testing agencies, clinical research organizations, and reproductive or family planning clinics. The Health Estate Insights and Relationship (HEIR) system introduces a multi-generational permission management service designed to solicit, store, and maintain the legal consents required to access, retrieve, and share health data among family members and authorized third parties. It supports granular control, enabling users to share all or part of their health data with one or many verified family members, while accommodating jurisdictional variations based on country, state, province, or territory.

A distinctive feature of the HEIR system is its ability to preserve and manage health data permissions across multiple generations, for both living and deceased family members. Users can designate a health data executor to manage or share a family member's health data in the event of death or incapacity, ensuring continuity of access for future generations. The system incorporates an intuitive graphical user interface (GUI) that facilitates the loading of health data into the system, assists with the collection of consents, and enables integration with external systems, databases, APIs, sensors, devices and web applications. Health data managed through the system may include, but are not limited to, diagnoses, treatments, medications, surgical history, diagnostic and genetic testing results, and behavioral or cognitive assessments.

The GUI also supports the discovery and verification of family member connections, as well as the assignment of authorized health data executors. Users can view their health data and selectively share portions with verified family members while retaining full control over what is disclosed. Privacy-preserving features such as anonymization and data masking allow users to share health data without compromising personal or familial identity. Users may revoke permissions, sever family data connections, or delete their account and associated data at any time, including data managed in their capacity as a health data executor.

Another defining aspect of the HEIR system is its integrated suite of data science models, algorithms, and analytics tools that enable the generation of predictive, descriptive, and prescriptive insights. With proper permissions, users can view summaries of their individual and family health data, identify and fill gaps in family health histories, filter out certain family relationships (e.g., non-biologically or genetically related members) from analyses, and generate a family-level biologic signature composed of genes, proteins, metabolites, and biomarkers. These insights are designed to inform disease risk assessment, personalized treatment, clinical trial eligibility, and fertility planning.

The system's Family Health Data Filtering Module intelligently scopes and tailors the data used in analytics based on the specific requirements of a given analysis. For example, if paternal lineage is irrelevant to a particular assessment, that subset of data can be excluded. HEIR also employs enrichment techniques to address gaps in individual or familial datasets, enhancing risk profiling and historical completeness. Additionally, the system ingests and compares family health data to diverse external reference sources such as clinical trial inclusion criteria, medication data, libraries of genetic and biologic data, fertility profiles, bio-imaging data, population health benchmarks and disease screening guidelines. This comparative capability enables the system to surface unique, family-level insights that may be overlooked in isolated individual health data analysis.

The analytics engine is capable of real-time monitoring and automatic updates of insights in response to changes in family health data, reference datasets, or underlying models and algorithms. Users may share health data and insights, either in full or in part, through various secure methods, including file transfer protocols (FTP), cloud storage with access controls, secure APIs, data exchange platforms, peer-to-peer channels, blockchain mechanisms, anonymized and masked data transmission, encrypted communication, authenticated user portals, and dedicated data repositories with controlled permissions.

Other aspects, embodiments and features of the system and methods of the present disclosure will become apparent from the following detailed description when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the device and method shown where illustration is not necessary to allow those of ordinary skill in the art to understand the device and method.

### BRIEF DESCRIPTION OF THE DRAWINGS

The preceding summary, as well as the following detailed description of the disclosed system and method, will be better understood when read in conjunction with the attached drawings. It should be understood, however, that neither the device nor the method is limited to the precise arrangements and instrumentalities shown.

FIG. 1 is an architecture block diagram of the Health Estate Insights and Relationship (Heir) System.

FIG. 2 is a logic flow diagram for connecting to, and verifying the authenticity of, a connection with another family member in the system.

FIG. 3 is a logic flow diagram depicting the process of requesting, appointing and transferring of health data executor appointments between users of the system.

FIG. 4 is a rending of the system's graphical user interface (GUI) for supporting the permissions required, by US state, to name a Health Data Executor.

FIG. 5 is a rendering of the system's graphical user interface (GUI), showing how a user (i.e., family member) can view their personal and family health data, derived insights, and information pertaining to other family member accounts for which they have been appointed as a health data executor.

FIG. 6 depicts how a family member, represented by PatientID X, can use the system's GUI to share all or part of their health data with another connected family member account, PatientID Y.

FIG. 7 is an end-to-end logic and data flow diagram showing key system components relating to the system's family health data filtering module, analytics engine, family health data gap detection and enrichment service, internal and 3^{rd} party analytics modules, as well as the system's data presentation layer, communication interface and services and modes of data sharing.

FIG. 8 is a diagram representing a system-generated, ‘Family Biologic Signature’ for PatientID 12200. This example of a Family Biologic Signature shows how several different types of data and values are consolidated into a single view a given PatientID, 12200, and their 1^{st}, 2^{nd} and 3^{rd} degree family member connections.

FIGS. 9A-9C illustrate how the system, with the appropriate permissions, can be configured to support the comparative analysis of a given PatientID's family health and biosignature data to: another unique PatientID/family member account (FIG. 9A), a group of PatientIDs/family member accounts (FIG. 9B), and one or many sets of reference and/or external data (FIG. 9C).

FIG. 10 is a rendering of the system's graphical user interface (GUI) where a family member can select from a menu of comparative analytics modules for viewing system-derived insights pertaining to clinical trial opportunities, family and fertility planning, personalized medicine and disease prevention strategies.

FIG. 11 is a diagram illustrating how the system's graphical user interface (GUI) works in concert with other system components (e.g., data presentation layer, communication interface, data services) to configure, transform, de-identify, encrypt, control access to, document and ensure compliance with industry health data standards.

### DETAILED DESCRIPTION OF SPECIFIC EMBODIMENTS

The subject matter of the present disclosure now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and example of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive.

Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list”.

Terms and phrases used in this document, and variations thereof, unless otherwise expressly stated, should be construed as open-ended as opposed to limiting. As examples of the foregoing: the term “including” should be read as mean “including, without limitation” or the like; the term “example” is used to provide exemplary instances of the item in the discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary, a variety of optional components are described to illustrate the wide variety of possible embodiments of the present invention.

A “computer” or “computing device” may refer to one or more apparatus and/or one or more systems that are capable of accepting a structured input, processing the structured input according to prescribed rules, and producing results of the processing as output. Examples of a computer or computing device may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

“Software” or “application” may refer to prescribed rules to operate a computer. Examples of software or applications may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Python, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). The program code may also be distributed among a plurality of computational units wherein each unit processes a portion of the total computation.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASHEEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G and the like.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested herein.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory or may be communicated to an external device so as to cause physical changes or actuation of the external device.

Herein, the terms “family member”, “user”, and/or “patient” are used interchangeably to denote any party, including family members, interfacing and/or interacting with a process for providing family health history data, and/or a person who is the subject of all, or part of, any health data/information obtained by a process for providing family health history data, and/or a legal guardian of a person who is the subject of any health data/information obtained by a process for providing family health history data, and/or an authorized agent of any party interfacing and/or interacting with a process for providing family health history data, and/or a person who is the subject of any health data/information obtained by a process for providing family health history data, and/or any other authorized party associated with any party interfacing and/or interacting with a process for providing family health history data, and/or a person who is the subject of any health data/information obtained by a process for providing family health history data.

Herein, the terms “family” and/or “family member” are used interchangeably to denote one or more genealogical relatives of an individual, regardless of remoteness and/or degree of separation from the individual.

Herein, the term “healthcare” includes any general and/or specialized treatment, assessment, maintenance, therapy, medication, and/or advice relating to all, or any portion of, a family member's, state of health, including but not limited to: general medical, specialized medical, surgical, dental, vision, psychological, and/or any other type of treatment, assessment, maintenance, therapy, medication, and/or advice.

Herein, the term “medical treatment” includes, but is not limited to: one or more medications and/or medication regimes; physical therapy; recommended dietary changes; recommended activity level changes; other lifestyle changes; and/or surgical procedures; and/or any prescribed and/or suggested regime, medication, treatment, activity, avoided activity, and/or program designed to improve, maintain, and/or slow the degradation of, a family member's health.

Herein the terms “health data”, “health information”, “health data”, “healthcare information”, and/or “health profile and healthcare history information” are used interchangeably to denote, but is not limited to an individual's medical data, health records, genetic and genomic data, imaging and diagnostic data, metabolic data, biomarker and biologic data, proteomic data, lifestyle and socioeconomic data and the like.

Herein the term “derived health profile and healthcare history information” and “derived health data” denote health profile and healthcare history information and/or data derived based, at least in part, on information contained in a family member's health data such as, but not limited to: patient data, medical health records, imaging and diagnostic data, biologic data, genomic data, proteomic data, metabolic data, lifestyle and socioeconomic data and the like; and any other health profile and medical history information capable of being derived from the family member's, health data, and of interest to the family member or another party.

Herein, the term “healthcare benefit program” and “health insurance plan” are used interchangeably to denote any policy, program, means and/or mechanism whereby a family member is provided benefits and/or service and/or entitlements to any form of healthcare.

Herein the terms “healthcare insurance plan providers” and “healthcare benefit program administrators” include but are not limited to: health insurance plan providers; health insurance plan administrators; healthcare expense account program providers; healthcare expense account program administrators; healthcare providers; employers; and/or any other parties generating and/or having access to a family member's health data.

Herein, the term “healthcare provider” denotes any individuals, persons, agencies, institutions, organizations, businesses, and/or other entities that provide medical treatment, medications, therapy, advice, and/or equipment. For example, herein, the term “healthcare provider” includes, but is not limited to: doctors; nurses; technicians; therapists; pharmacists; counselors; alternative medicine practitioners; all medical facilities; doctor's offices; hospitals; emergency rooms; clinics; urgent care centers; alternative medicine clinics/facilities; physical therapy clinics/facilities; and any other party, entity, and/or facility providing general and/or specialized treatment, assessment, maintenance, therapy, medication, and/or advice relating to all, or any portion of, a family member's state of health, including but not limited to, general medical, specialized medical, surgical, dental, vision, psychological, and/or any other type of treatment, assessment, maintenance, therapy, medication, and/or advice.

Herein, the term “Health Data Executor” denotes any individuals, persons, family members, relatives, healthcare providers, attorneys, agencies, institutions, organizations, businesses who have the legal authorization to access all or part of a patient's health data for a specified period of time.

Herein, and in the context of the Health Estate Insights and Relationship (HEIR) system, the term ‘Family-level Biologic Signature” refers to a measurable set of biological markers, consolidated for one or many family members, such as genes, proteins, metabolites, or imaging patterns that provide insights into a person's health status, disease presence, or response to treatment. These signatures can be derived from various sources, including blood tests, genomic sequencing, imaging scans, and wearable health sensors.

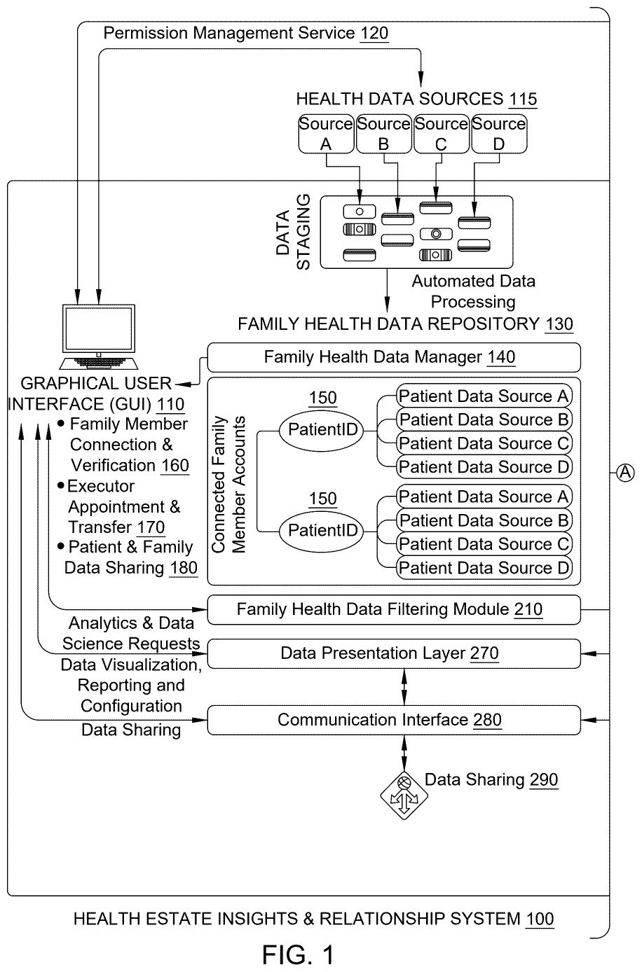

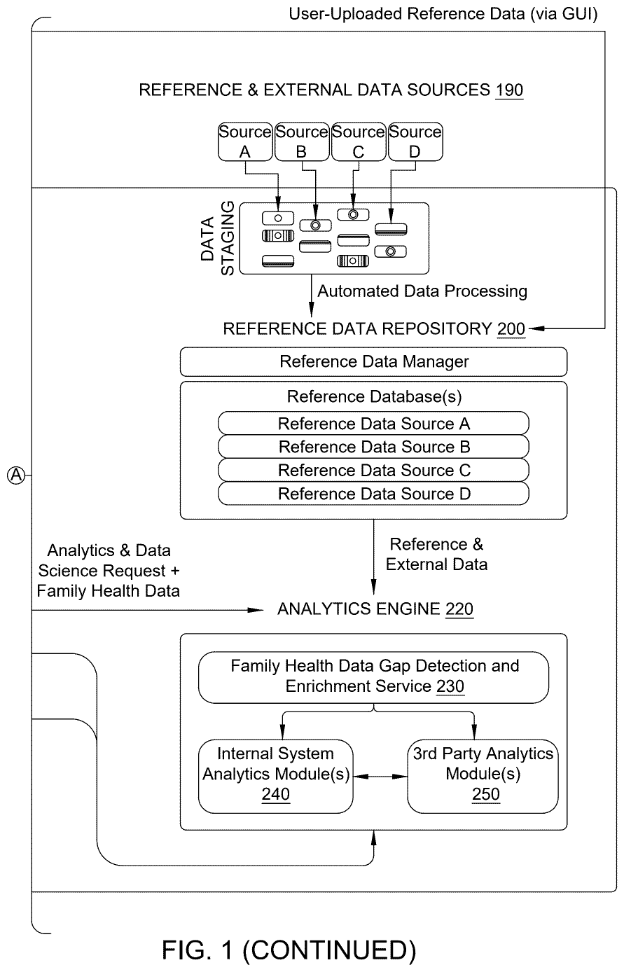

The Health Estate Insights and Relationship (HEIR) System (100), as illustrated in FIG. 1, provides a comprehensive and extensible architecture for managing, sharing, and analyzing individual and family health data. Primary users of the system (100) include, but are not limited to, family members, legal guardians of family members, designated healthcare providers, and authorized private or government agencies with jurisdiction over a family member's data. Access to the system (100) may occur through various devices, including desktop or laptop computers, tablets, smartphones connected via secure applications and networks, mobile applications, integrations with other systems and third-party platforms, and through connected devices such as wearables and sensors.

Central to the system is a graphical user interface (GUI) (110), which is configured to support multiple user categories and roles. In various embodiments, the GUI (110) enables family members to manage both their individual and family health data. Additionally, the GUI (110) integrates with approved third-party individuals, organizations, applications, devices, sensors, and systems to facilitate the sharing, analysis, and reporting of health data and derived insights.

The GUI (110), in communication with the system, allows users to search for personal health data residing in various data sources (115), which may include: other family members, legal guardians, healthcare providers, government agencies, electronic medical record (EMR) or electronic health record (EHR) systems, insurance providers, benefits administrators, financial institutions, genetic testing organizations, reproduction and family planning centers, digital health devices, sensors, and any other repositories of medical, clinical, claims, biologic, genetic, genomic, epigenetic, proteomic, metabolic, microbiome, imaging, physiological, lifestyle, insurance, or financial data.

In one or more embodiments, the system (100) includes a multi-generational permission management service (120), which works in conjunction with the GUI (110) to help users solicit and manage the necessary consents for accessing and transferring health data in accordance with jurisdiction-specific regulations (e.g., country, state, province, or territory). Once access is granted, the system supports the secure transmission of health data in various formats, which are depicted with distinct patterns in FIG. 1.

The system also comprises a Family Health Data Repository (130) for securely storing the health data associated with one or more family member accounts. A Family Health Data Manager (140) processes, organizes, and reconciles incoming data from disparate source systems. Each family member account is uniquely identified using a system-generated PatientID (150), which links all associated health data. FIG. 1 shows how, with proper permissions, two or more family member accounts can be connected via their respective PatientIDs (150).

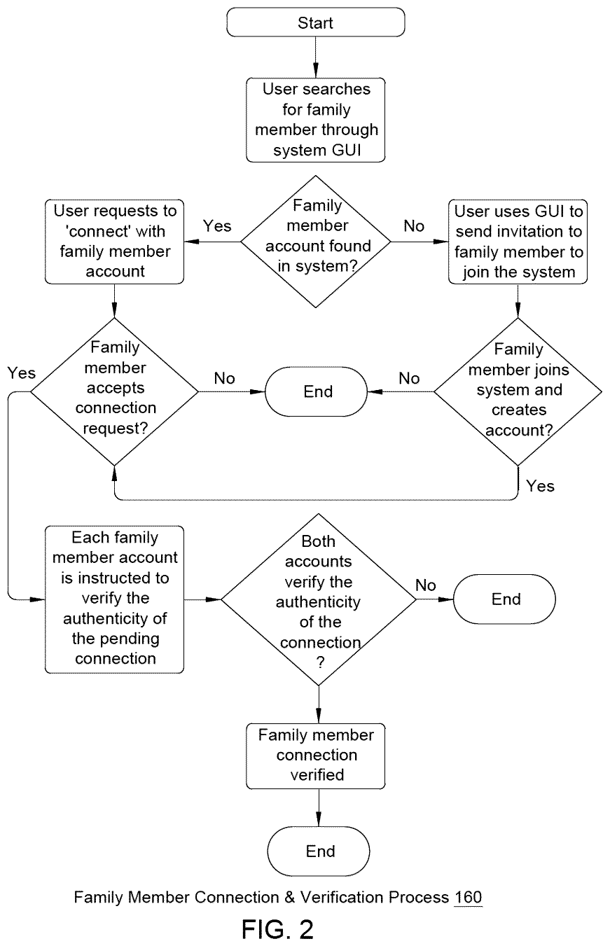

FIG. 2 illustrates the Family Member Connection and Verification process (160), which is used to search for and invite family members, issue and accept connection requests, and verify the authenticity of connections. This process, executed via the GUI (110), also allows users to revoke connections at any time.

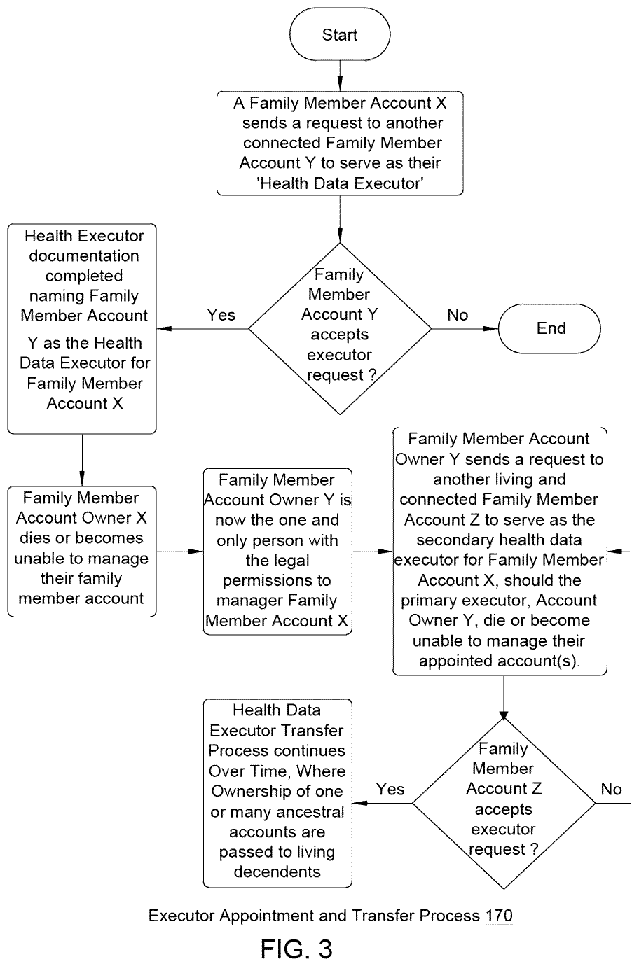

The Health Executor Appointment and Transfer process (170), shown in FIG. 3, enables users to request, accept, and complete the documentation necessary to serve as a health data executor—i.e., a legally authorized individual who may access and manage the health and medical records of a family member. The process also supports the transfer of executor responsibilities to another verified family member account if the original account owner has died or become incapacitated, thereby enabling authorized descendants to manage legacy health data.

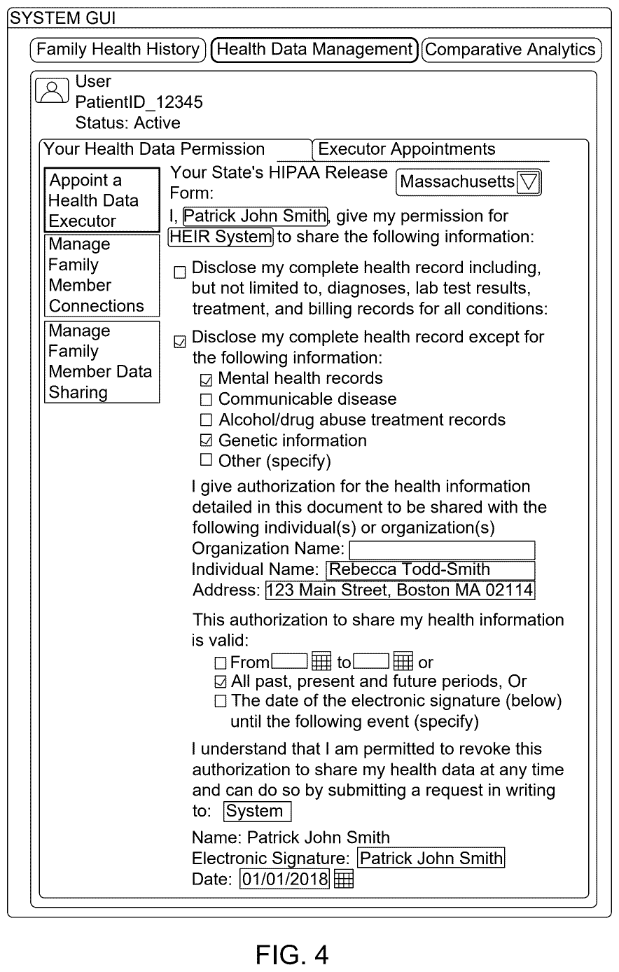

As depicted in FIG. 4, the GUI (110) can also be used to solicit, manage, and store HIPAA Release Forms, allowing users to share some or all of their health data with another individual or organization. Users can select HIPAA forms based on the state of data ownership and designate the specific time frame and scope of access. The system stores and archives all documentation and electronic signatures related to executor appointments and data-sharing permissions.

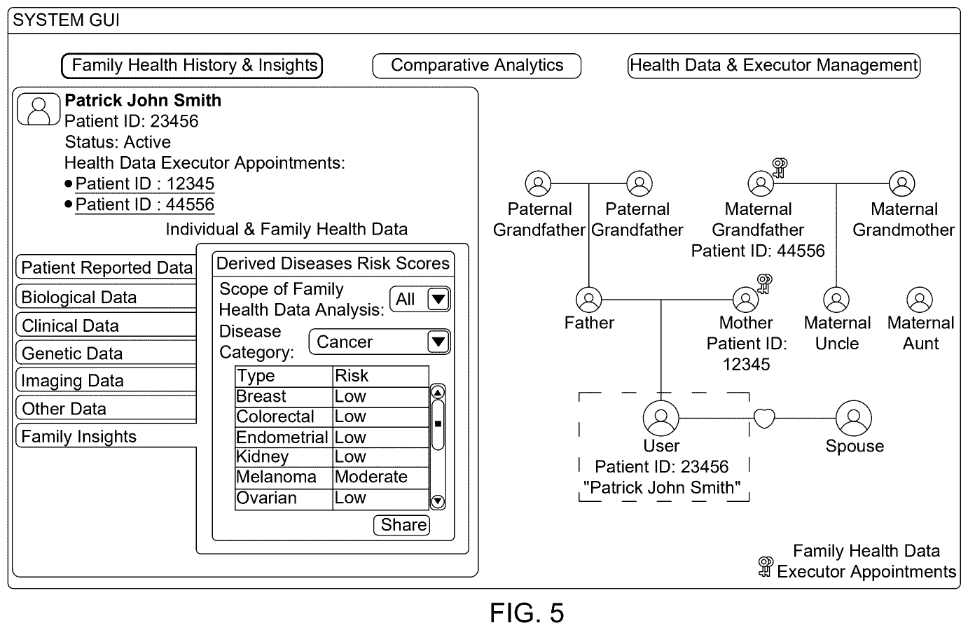

In the example shown in FIG. 5, a user (Patrick John Smith) has been appointed as the health data executor for two other PatientIDs (150): 12345 and 44556. The GUI (110) displays a unified view of the user's personal and family health data, including cancer-related insights derived from the connected family data. The user, acting as executor, has full access to the shared health data of the connected PatientIDs (150).

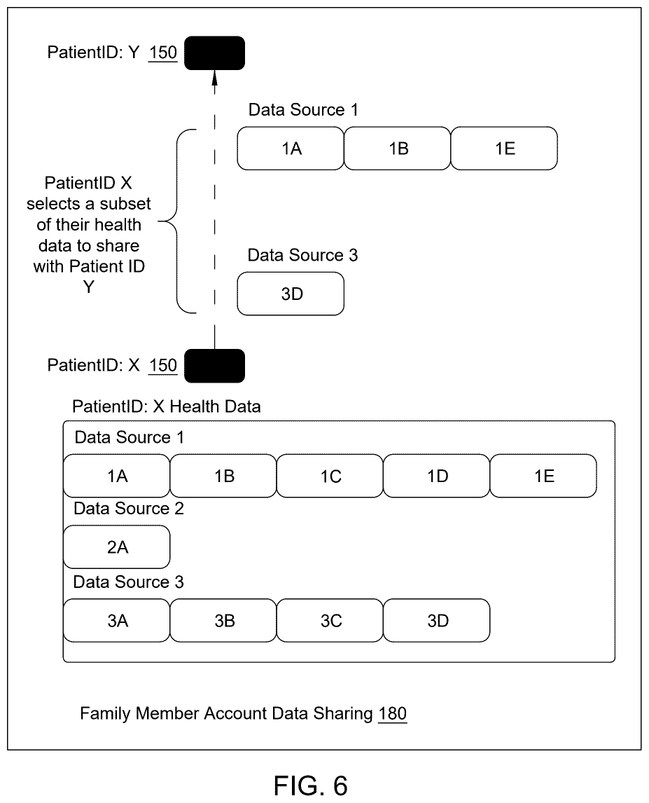

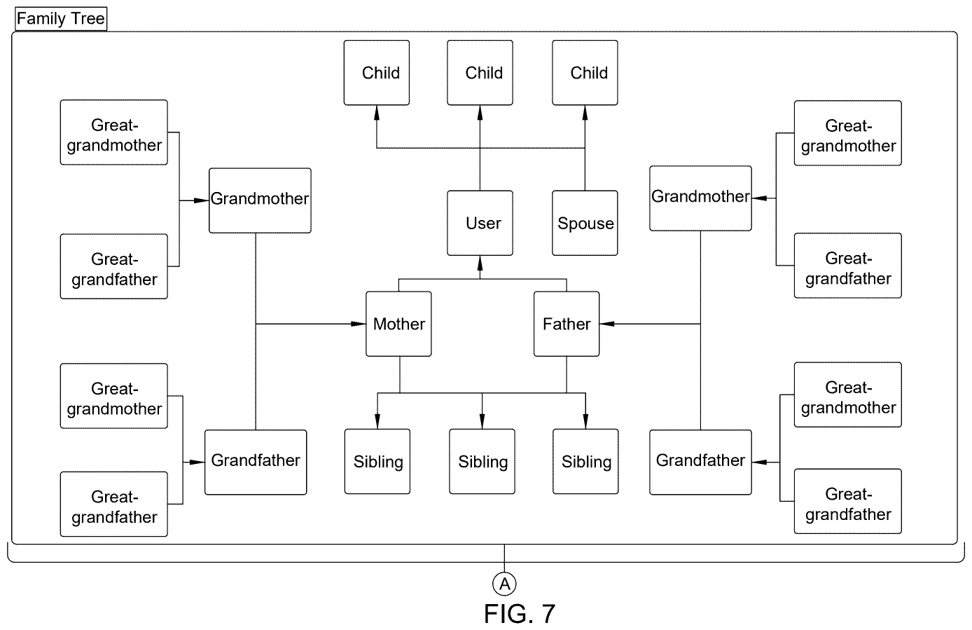

FIG. 6 further illustrates how the system enables selective data sharing between family member accounts. A user associated with PatientID X (150) is connected to three distinct data sources: Data Source 1, Data Source 2, and Data Source 3. Each data source contains different types of health data, represented by alphanumeric codes (e.g., 1A, 1B, 1C), including but not limited to: EHRs, claims, surveys, genetic and proteomic data, biomarkers, imaging, wearable data, clinical trial records, and physician notes. In the scenario shown, PatientID X shares data from Data Source 1 and Data Source 3—but not from Data Source 2—with PatientID Y (150).

Returning to FIG. 1, the system is also capable of ingesting, processing, and storing external and reference data (190) into a dedicated Reference Data Repository (200). These data are used to support advanced analytics, modeling, and other system services.

Examples of reference and external data (190) include clinical trial eligibility data, genomic and genetic profiles, personalized medicine labels, fertility and family planning information, health risk guidelines, population benchmarks, billing codes, biologic signatures, and insurance coverage details. In various embodiments, family members can upload such data directly via the GUI (110), including diagnostic test results, biomarker profiles, imaging reports, and family planning documentation. To account for the wide variety of reference datasets, the system (100) includes configurable tools to manage the ingestion, processing, and storage of such data according to user-defined preferences and system standards.

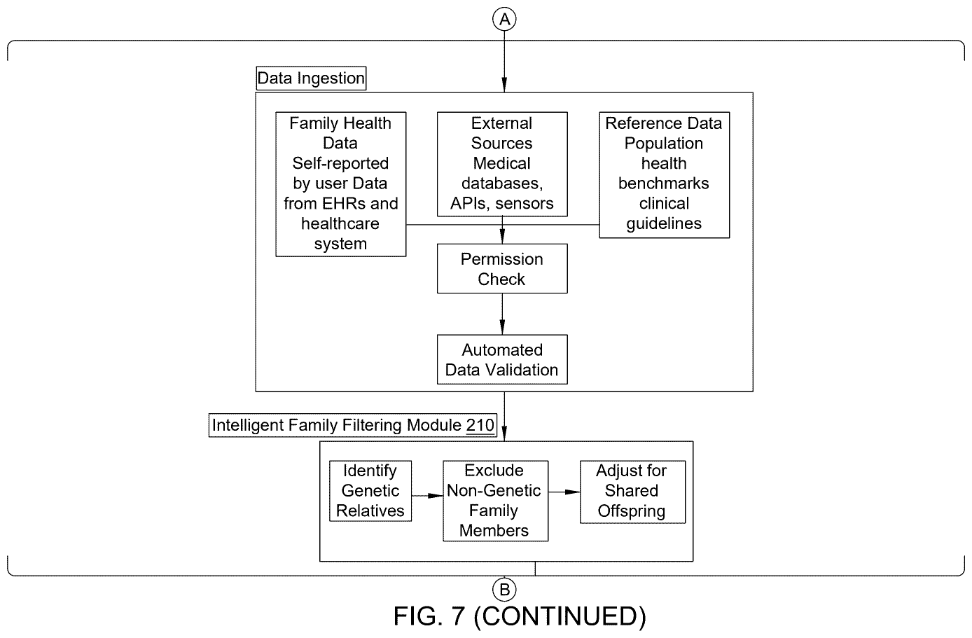

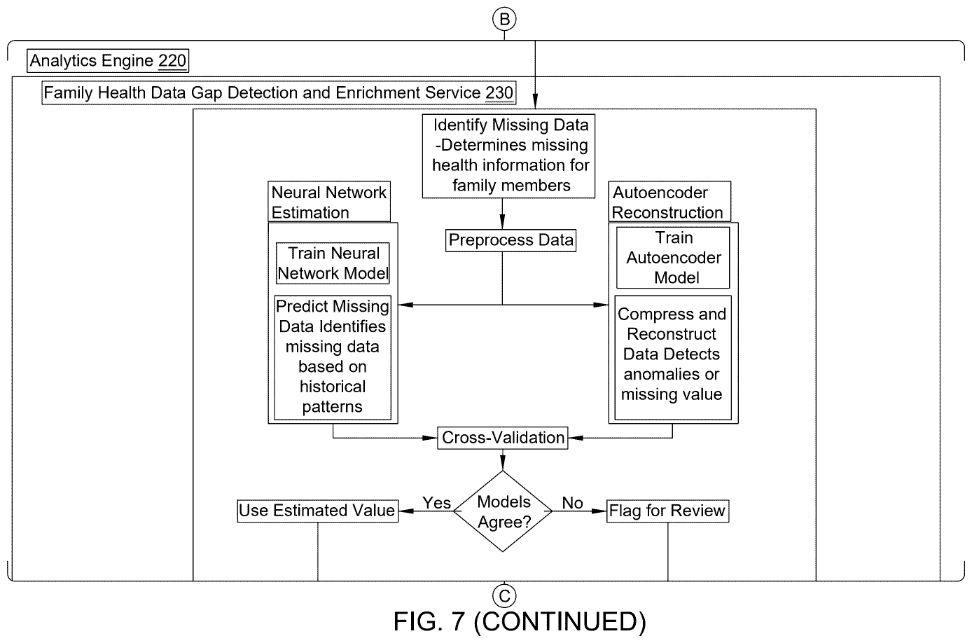

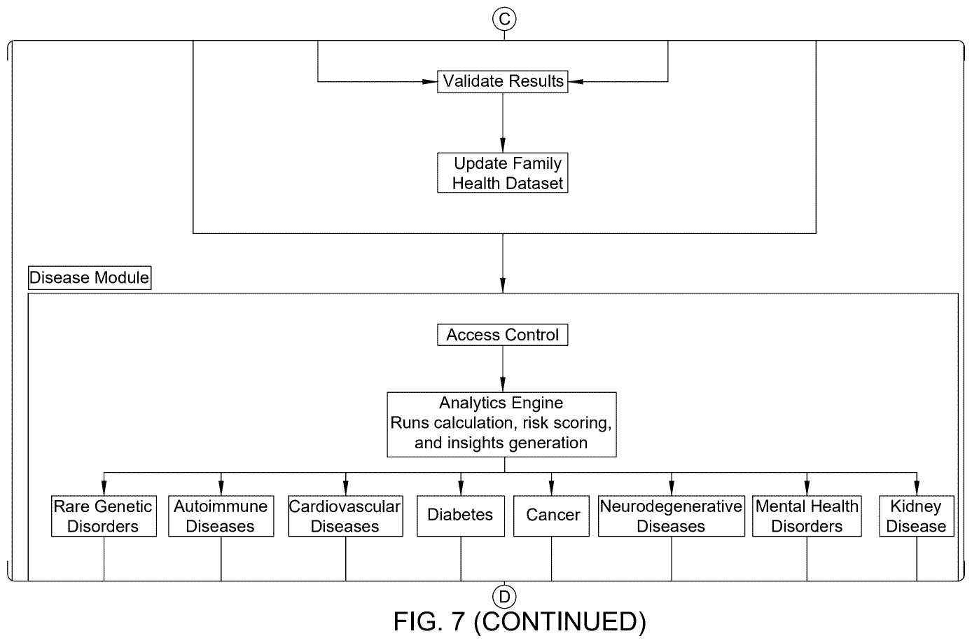

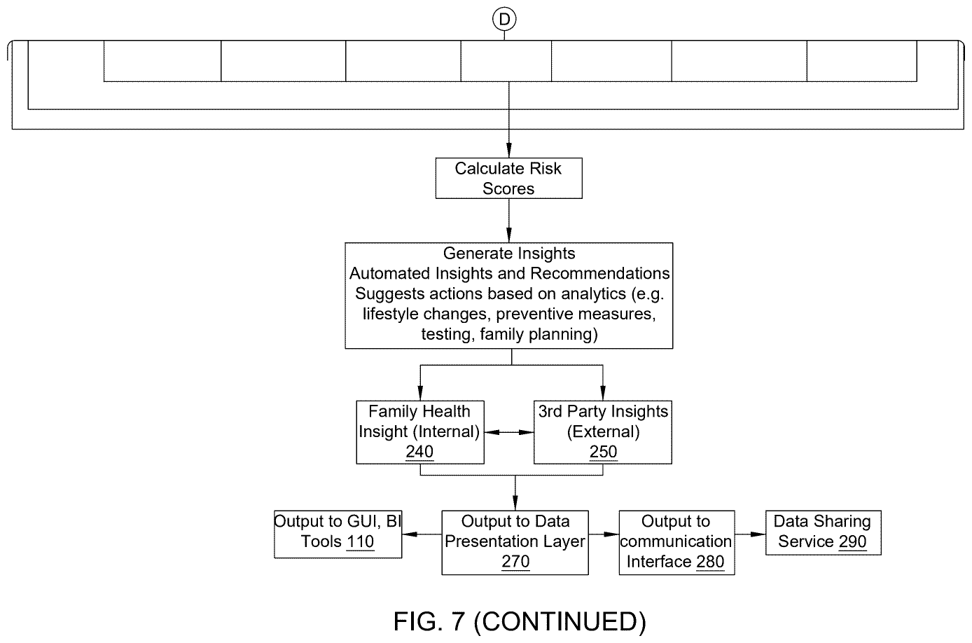

Health Estate Insights and Relationship (HEIR) System Analytics. As depicted in FIG. 7, the HEIR System (100) includes a suite of analytics components and sub-modules designed to load, authenticate, process, and analyze individual health data, family health data, and reference or external data. Central to these analytics capabilities is the system's Family Health Data Filtering Module (210), which enables the intelligent selection and organization of relevant family health data for inclusion in analyses, calculations, and predictive modeling.

The Family Health Data Filtering Module (210) performs several key functions. It supports family structure analysis by using data clustering techniques to analyze the relationships between a given PatientID (150) and its connected family members, distinguishing biologically and genetically related individuals. It also enables the selective inclusion or exclusion of all or some of an individual's biologically and/or genetically related family members based on configuration settings, ensuring that health analytics remain relevant and personalized to the selected PatientID. Moreover, the module facilitates the intelligent inclusion of shared offspring data, allowing health data from non-biologically related family members (e.g., spouses) to be indirectly considered for child-specific analytics when shared biological children exist. For instance, a spouse's health data may be relevant for analyses concerning their children but excluded from analytics concerning the primary PatientID. The module is also capable of dynamic filtering, adapting the inclusion criteria of family member health data based on the context and purpose of the analysis, such as distinguishing between individual risk prediction and multi-generational trend analysis. These functions ensure that all predictions and insights generated by the system are biologically, genetically and contextually appropriate.

FIG. 7 further illustrates the system's Analytics Engine (220), which in certain embodiments includes multiple modules: the Family Health Data Gap Identification and Enrichment Service (230), Internal Analytics Modules (240), and Third-Party/External Analytics Modules (250). These components collectively enable comprehensive health analytics and insight generation across individual and family data sets.

The Family Health Data Gap Identification and Enrichment Service (230) is designed to identify and fill gaps in both individual health records and broader family health histories. This includes missing data related to a specific PatientID (150) as well as incomplete records within that PatientID's network of connected family members. The enrichment process draws on internal system data, verified family health data, and external reference datasets to improve completeness and analytic value.

In certain embodiments, the gap analysis process within the Gap Identification and Enrichment Service (230) follows a structured and multi-step approach. First, data preprocessing is performed to clean, normalize, match and structure incoming health data. This includes removing duplicate entries, resolving formatting issues, and applying transformation techniques to ensure data interoperability. Next, the system conducts missing data detection, using statistical and machine learning techniques to identify absent data elements such as unreported conditions, genomic markers, or data from deceased or inaccessible family members.

Once gaps are detected, the system applies the Family Health Data Filtering Module (210) to include only biologically and/or genetically relevant family data in subsequent analyses. The system then engages in neural network-based estimation, utilizing trained models based on complete data from verified family member connections to infer missing data using familial, genetic, and medical correlations. Complementing this, the system also applies autoencoder-based reconstruction, a deep learning method that compresses available health data into a latent space and reconstructs likely missing data elements based on probabilistic modeling.

To ensure accuracy, the system performs cross-validation of estimates, comparing outputs from the neural network and autoencoder models against each other and applying confidence thresholds to assess consistency. If the outputs align and meet predefined accuracy criteria, the system proceeds to final data validation and integration, at which point the estimated values are incorporated into the Family Health Data Repository (130). In cases where there is a conflict between model outputs or if thresholds are not met, the system flags the data for manual review through the system's GUI (110), preserving transparency and user oversight in the enrichment process.

Descriptive, Predictive, Prescriptive, and Comparative Analytics. In addition to the Family Health Data Gap Detection and Enrichment Service (230), the HEIR System's

Analytics Engine (230), as illustrated in FIG. 7, supports a wide range of analytics functions across individual and family datasets. This includes descriptive analytics, which generate summaries of a user's health data encompassing demographic details, vitals, lab results, diagnoses, medical claims, hospitalizations, surgeries, medications, genetic testing outcomes, prevalent biomarkers, bio-imaging, physiological trends, and more. In certain embodiments, the system's event-driven architecture supports real-time health monitoring by continuously tracking biometric and physiological changes for a given PatientID (150).

The Analytics Engine (230) supports both internal analytics modules (240) and third-party/external analytics modules (250), enabling diverse data science operations, including predictive modeling. Examples of predictive analytics include the calculation of health risk scores for specific diseases and inherited conditions, such as autoimmune disorders, cancer types, cardiovascular diseases, diabetes, mental health disorders, neurodegenerative conditions, and rare or inheritable diseases. These predictions are tailored by leveraging both individual and family-level data within the HEIR System.

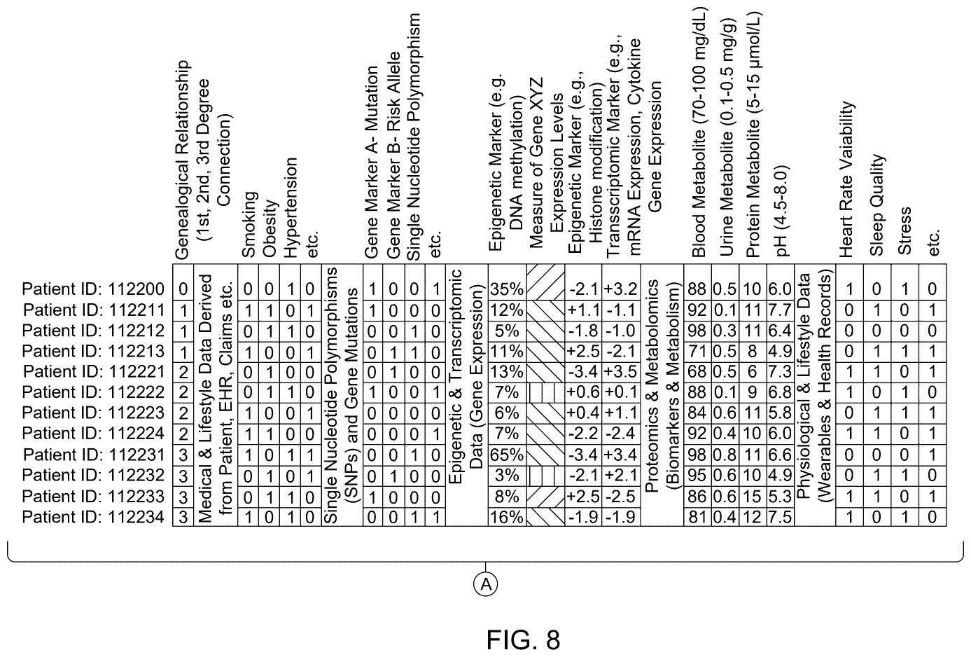

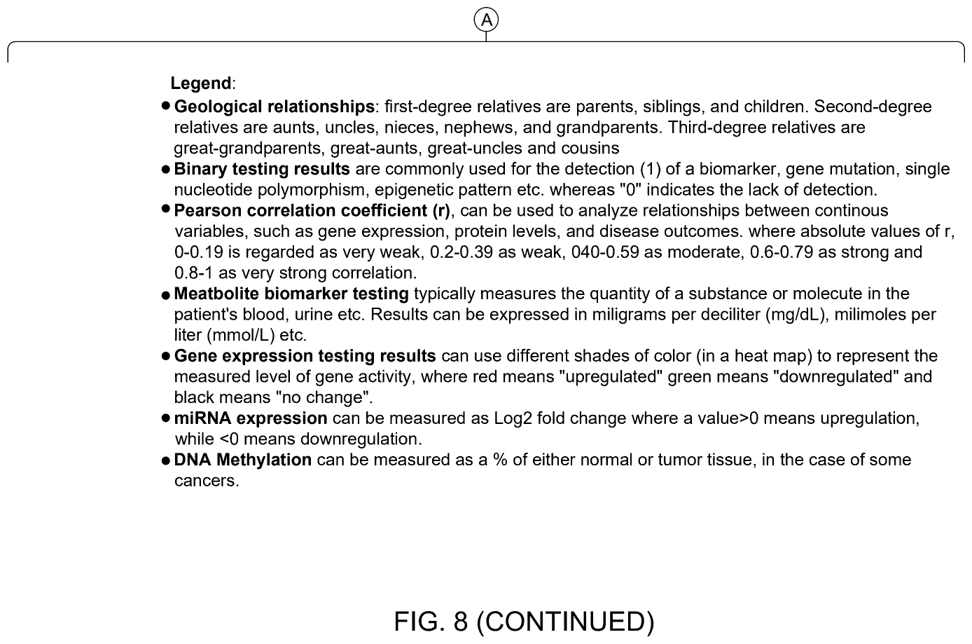

Family-Level Biologic Signatures. As shown in FIG. 8, the system can generate a Family-Level Biologic Signature (260), which consolidates biologic and health data across multiple connected PatientIDs (150). These biologic data may include genes, proteins, metabolites, biomarkers, cell signaling pathways, disease indicators, genetic mutations, molecular profiles, immune markers, image-based data, and other biological signatures. Unlike traditional patient-level profiles, family-level biologic signatures incorporate multigenerational data, thereby enhancing the system's predictive capabilities in areas such as disease risk modeling, drug response prediction, treatment planning, and personalized medicine.

In FIG. 8, PatientID 112200 represents the target individual whose Family Biologic Signature (260) is derived from both direct data and data from connected first-, second-, and third-degree relatives. The Analytics Engine (230) uses the consolidated signature to derive insights related to the effectiveness and tolerability of personalized treatments, compatibility of egg or sperm donors, identification of relevant clinical trials, and strategies for disease prevention and screening.

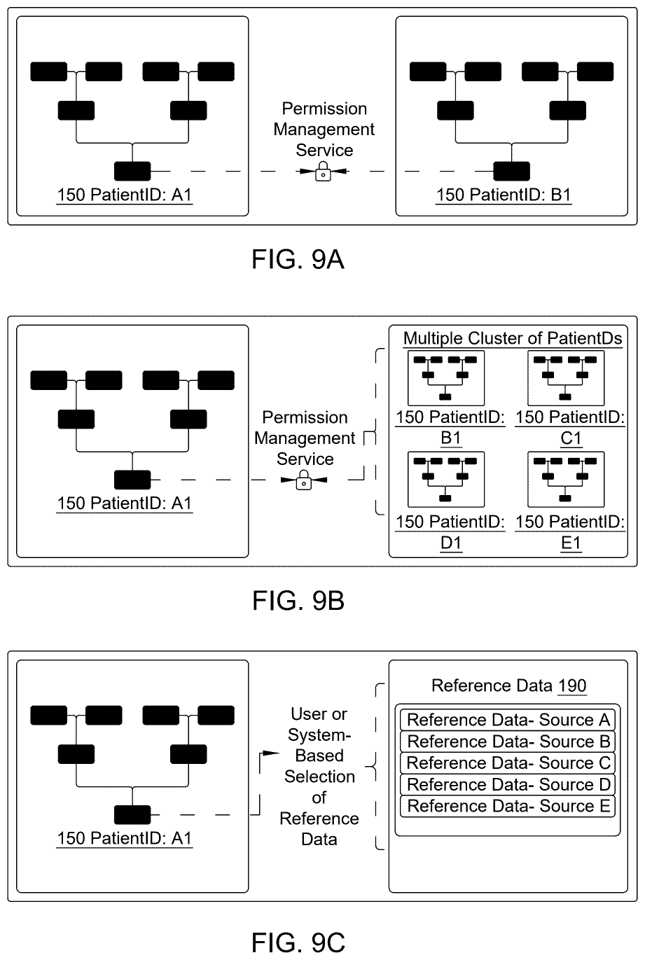

Comparative Analytics. FIG. 9 illustrates how, with appropriate permissions, the system can support comparative analytics between a given PatientID's family health and biologic data and that of another PatientID (FIG. 9a), a cohort of PatientIDs (FIG. 9b), or one or more external reference data sources (190) (FIG. 9c). These comparative capabilities allow users and authorized professionals to evaluate individual and family health risks in context with peers, reference populations, or donor profiles.

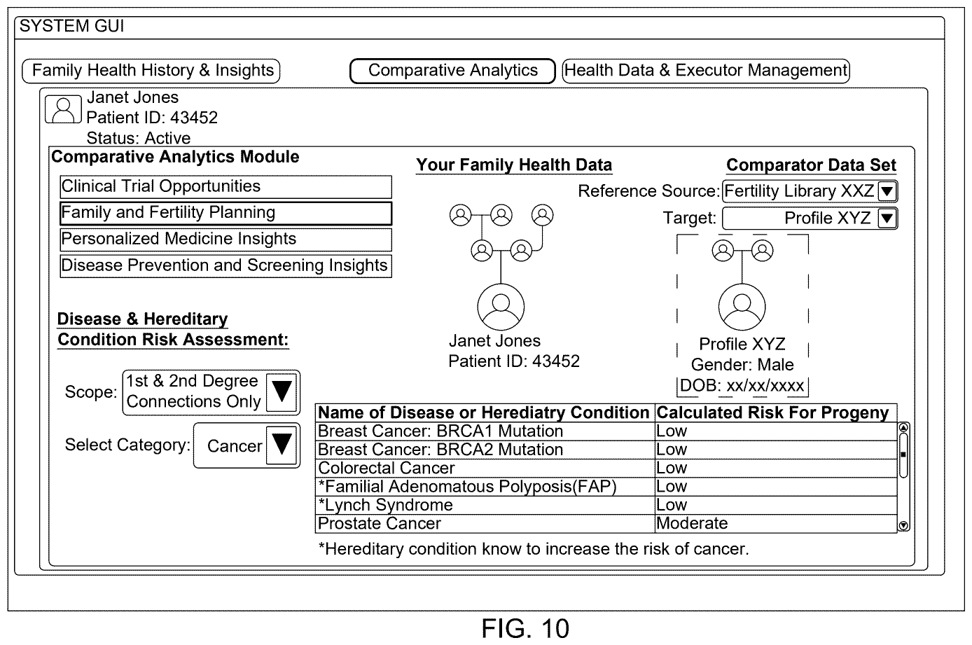

In FIG. 10, a family member (Janet Jones) uses the system's GUI (110) to navigate a menu of comparative analytics modules. The user selects a prospective fertility profile (e.g., Profile XYZ) and compares her family's health data against it, specifically reviewing cancer-related risk scores for hypothetical offspring. This interaction demonstrates how comparative analytics can be configured to support fertility planning, clinical trial matching, personalized medicine, and other use cases.

Data Presentation and Visualization. Referring back to FIG. 1, the system includes a Data Presentation Layer (270) that transforms analytic outputs from the internal (240) and third-party (250) modules into various formats. These include data visualizations (e.g., dashboards, graphs, charts), export-ready formats (e.g., CSV, Excel, PDF, JSON), business intelligence-compatible formats (e.g., Power BI, Tableau), embeddable datasets and reports, and customizable configurations for end-user-designed reports and exports. The Data Presentation Layer (270) ensures real-time synchronization with the Analytics Engine (230) so that updates are reflected dynamically.

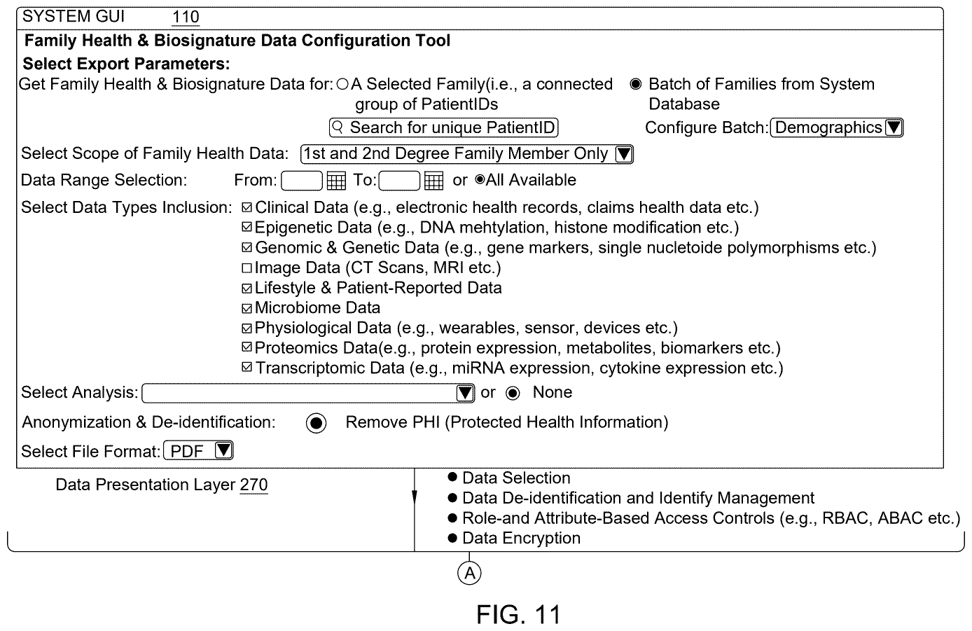

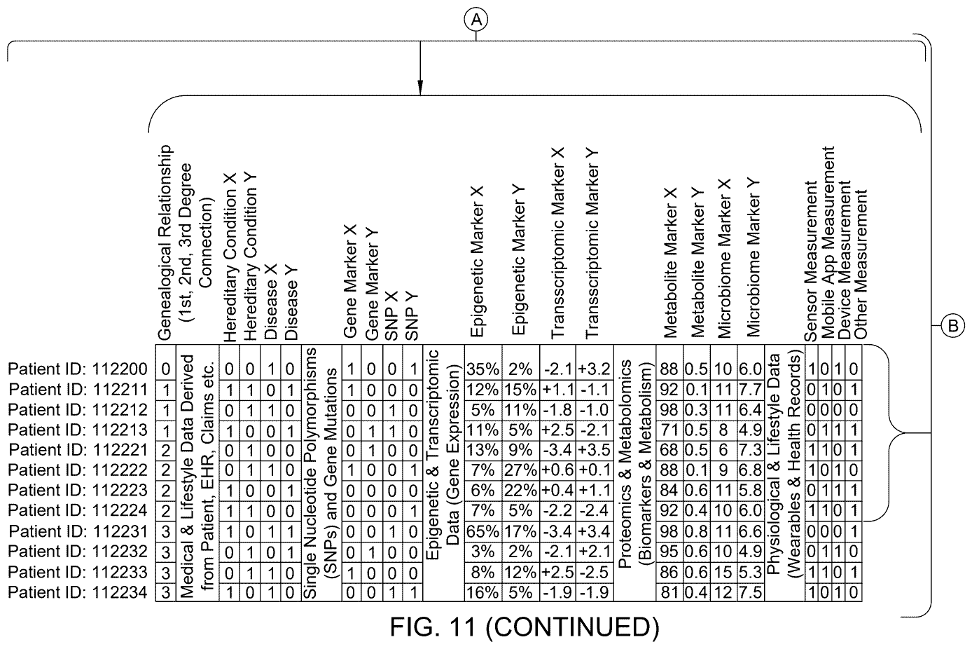

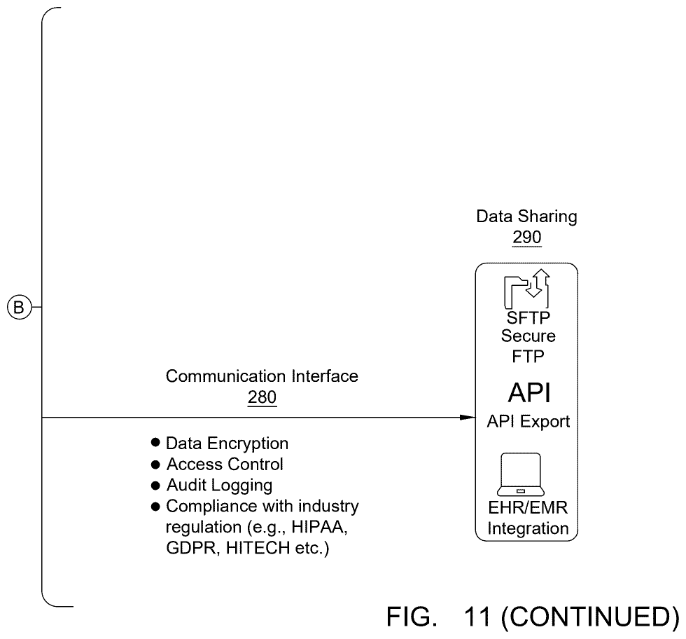

FIG. 11 shows how the GUI (110), in communication with the Data Presentation Layer (270) and the system's Communication Interface (280), supports the configuration of personalized data views, exports, and reports. These components work together to enable functionalities such as data selection, transformation, encryption, and de-identification; role-based and attribute-based access control (RBAC/ABAC); federated identity management (FIM); audit logging; and compliance with data protection regulations including HIPAA (U.S.), GDPR (EU), and HITECH.

The system can generate various types of reports and insights, including individual and family health summaries, gap analysis reports with estimated data and confidence scores, donor compatibility rankings, personalized treatment recommendations, clinical trial eligibility reports, disease prevention suggestions, and real-time notifications related to significant risk updates or data changes. The recommendations may include, but not limited to, family and reproductive plannings, disease prevention strategies, clinical trial and personalized medicine recommendations.

Privacy and Data Control. In certain embodiments, and as depicted in FIG. 1, the system allows users to manage privacy and data-sharing preferences directly through the GUI (110). Users can selectively identify which parts of their individual or family health data are shared with connected family members or approved third parties, including healthcare providers, organizations, and government agencies. No data are shared without explicit permission and validation of the target source. Users can define the duration of data sharing, after which access is automatically revoked and the data are securely deleted. Additional controls enable the de-identification or anonymization of shared data, revocation of previously granted access or connections, deletion of specific data elements, or deletion of an entire PatientID account.

Data and Insights Sharing. Finally, the system's Communication Interface (280), as shown in FIG. 1, facilitates secure sharing of data and insights (290) through a variety of methods. These include secure APIs, file transfer protocols such as SFTP and FTPS, HTTPS-based exchanges, virtual private networks (VPN), direct secure messaging (DSM), secure messaging platforms, health information exchange (HIE) protocols, HIPAA-compliant cloud storage (e.g., AWS, Azure, Google Cloud, Snowflake), and integrations with approved third-party applications, devices, and sensors. The Communication Interface (280) ensures that health data and derived insights are shared securely, in compliance with regulatory standards, and only with validated recipients.

The following provides a more detailed discussion of various aspects of the invention, including the permission management framework, executor appointment and transfer functionality, family health data filtering processes, and related analytics features. These components work in concert to enable secure, configurable, and legally compliant intergenerational access to health data, while supporting biologically, genetically and contextually relevant analysis and dynamic data sharing based on user-defined preferences and jurisdictional requirements.

As shown in FIG. 1, the HEIR System (100) comprises a modular architecture that enables secure, configurable, and legally compliant intergenerational management of health data across a network of related individuals. Central to the system is a data repository (130), configured to store diverse types of health data associated with multiple family member accounts. These data include but not limited to: electronic health records, genetic testing results, physiological and biologic data, and self-reported health information, all of which may be ingested from internal or external sources via the communication interface (280) and managed through the system's graphical user interface (110). Each family member account is associated with a unique PatientID (150), enabling structured, account-specific data organization and permission tracking.

In communication with the data repository (130) is the system's permission management module (120), which facilitates the collection, storage, and enforcement of user consents and legal documentation necessary for accessing and sharing health data. These consents may include HIPAA release forms or equivalent legal instruments depending on jurisdiction. The module supports a highly configurable user workflow through the GUI (110), where users can upload or digitally execute consent forms, assign access permissions, define temporal access windows, and designate one or more individuals as health data executors. These executors are granted access rights to another family member's health data in the event of death, incapacity, or another predefined triggering event. As illustrated in FIG. 3, the health executor appointment and transfer process (170) involves system-verified handoff of access control from the original data owner to a verified and connected family member account. All legal documentation related to executor transfers is securely stored and auditable within the permission management system.

The system further includes a communication interface (280), configured to securely interface with external sources such as healthcare provider systems, health information exchanges, insurance databases, and diagnostic platforms. With proper permissions stored in the permission management module (120), the system can retrieve, update, and synchronize health data based on the user's evolving health status or institutional record availability. Secure APIs, file transfer protocols, direct messaging standards, and HIPAA-compliant cloud storage providers (e.g., AWS, Azure) are supported modes of transfer, as depicted in FIG. 1.

To facilitate biologically, genetically and contextually relevant health analytics, the HEIR System incorporates a Family Health Data Filtering Module (210), shown in FIG. 7. This module is configured to identify and classify family member connections, including first-, second-, and third-degree relatives, based on relationship metadata stored within or linked to the system's accounts. As illustrated in FIG. 2, users can establish, verify, and revoke family member connections through the GUI (110). The filtering module supports fine-grained control over which family member data are included in system-based analyses, enabling inclusion or exclusion based on biological relatedness, jurisdictional rules, or user-defined criteria. For example, as shown in FIG. 6, the system may include health data from a non-biologically related spouse in offspring-related analyses if the spouse shares biological children with the user but exclude that same data from risk predictions focused solely on the user's own profile. The module also supports dynamic filtering based on the purpose of analysis—for instance, narrowing focus for personalized risk prediction versus broader inclusion for multi-generational health trend modeling. Clustering algorithms and relationship graph analysis are employed to identify relevant relational paths and generate biologically, genetically and contextually appropriate data subsets.

The family health data filtering module (210) is tightly integrated with the system's Analytics Engine (230), which comprises internal analytics modules (240), external analytics modules (250), and a Family Health Data Gap Identification and Enrichment Service (230). The gap detection process, shown in FIG. 7, includes standardizing and preprocessing incoming data, applying machine learning-based techniques such as neural network estimation and autoencoder reconstruction to identify and infer missing data elements, and cross-validating those estimates for confidence before integration into the family health dataset. This enriched dataset is then used to support downstream analytics.

The system supports a variety of analytics functions, including descriptive, predictive, prescriptive, and comparative analytics. Descriptive analytics generate summaries of individual and family health data, including demographics, lab results, genetic findings, diagnoses, medications, and more. Real-time physiological monitoring is supported via event-driven updates from sensors or wearables. Predictive analytics modules estimate disease risks, identify inherited condition likelihoods, and evaluate treatment compatibility using consolidated family data. As shown in FIG. 8, the system can generate a Family-Level Biologic Signature (260) by aggregating biologic data, such as genes, proteins, biomarkers, and cellular pathways, across multiple connected PatientIDs (150). This biologic signature may be used to assess the effectiveness of personalized medicine, donor compatibility for reproductive planning, or to match patients to clinical trials.

As depicted in FIG. 9, the system supports comparative analytics wherein a PatientID's family health and biologic data may be compared to (FIG. 9a) another

PatientID's dataset, (FIG. 9b) a cohort of users, or (FIG. 9c) a curated set of external reference datasets (190). In the example of FIG. 10, a user (Janet Jones) compares her family's cancer risk scores to prospective fertility donor profiles, selecting the source and scope of data to be included in the analysis.

The analytics results are displayed and managed through the system's Data Presentation Layer (270), which transforms raw outputs into charts, dashboards, exportable files (e.g., CSV, Excel, PDF), and embeddable reports. The presentation layer supports integration with external BI tools (e.g., Tableau, Power BI), internal apps, and custom data visualization environments. The GUI (110), working in concert with the presentation layer (270) and communication interface (280), also enables users to configure custom reports, apply de-identification or anonymization procedures, enforce access controls using RBAC, ABAC, or federated identity management, and generate compliance-ready outputs aligned with standards such as HIPAA, HITECH, and GDPR, as illustrated in FIG. 11.

Users may manage privacy settings through the GUI (110), defining which health data to share, for how long, with whom, and under what conditions. Access can be selectively granted, revoked, or expired based on time-based or event-based triggers. Permissions may include restrictions by data type (e.g., diagnoses vs. genetic results), recipient (e.g., family vs. third-party), and level of identifiability (e.g., fully anonymized vs. de-identified). The system enforces these policies through integration between the permission management module (120), the filtering module (210), and the communication interface (280), ensuring that data and insights (290) are only shared in accordance with the user's express preferences and legal constraints.

This integrated architecture and process enable a powerful yet flexible framework for managing intergenerational health data access, executor-driven permissions, biologically and genetically contextualized analytics, and legally compliant data sharing, ensuring continuity and control over sensitive health information across time, relationships, and jurisdictions.

The following provides a detailed discussion of the invention's gap detection and enrichment functionality, as well as the generation and application of a family-level biologic signature, including the associated data structures, analytics processes, and system modules involved in these features.

As illustrated in FIG. 7, the system includes a gap detection module and enrichment engine configured to address missing, incomplete, or unreported health data across a user's individual health records and those of their connected family members. The system stores a wide array of health data, including electronic medical records, genetic and genomic data, biologic markers, and user-reported information. The gap detection module identifies deficiencies within this dataset, including but not limited to: missing disease history, unreported lab or imaging results, incomplete biomarker panels, or gaps resulting from deceased or inaccessible relatives. A preprocessing pipeline cleans, normalizes, matches and structures the data prior to analysis to ensure compatibility and remove redundancies.

Once gaps are detected, the system initiates an enrichment process using one or more machine learning models. In certain embodiments, these include a neural network trained on system-based family health data and/or data deriving from external sources, and an autoencoder trained to compress existing data into a latent representation and reconstruct missing values with high probability estimates. The system compares the outputs from these models using a cross-validation module. Where both models produce consistent results that meet a predefined confidence threshold, the estimated data are automatically incorporated into the Family Health Data Repository (130). In cases where discrepancies exist or confidence is insufficient, the system flags the estimates for manual review via the graphical user interface (110), allowing the user or a healthcare proxy to validate or reject the suggested enrichment.

To ensure that enrichment and analytics remain biologically, genetically and contextually relevant, the system incorporates the Family Health Data Filtering Module (210), which evaluates relationship metadata and filters family member accounts based on degrees of relatedness (e.g., first-, second-, or third-degree relatives) and biologic relevance. In some cases, the system may include non-biologically related individuals in certain analyses if they share biological children with the user, particularly when evaluating inherited traits or risk factors related to offspring.

In support of advanced analytics, the system includes functionality for generating a family-level biologic signature, as shown in FIG. 8. This biologic signature is a composite representation of biologic and health data derived from the user account and one or more connected family member accounts. Biologic data contributing to the signature may include, but are not limited to, genetic sequences, gene expression profiles, protein markers, metabolite panels, biomarkers, cell signaling pathways, immunologic features, and even image-derived biologic traits. The biologic signature is filtered using the Family Health Data Filtering Module to ensure that only biologically and/or genetically relevant relatives contribute to the dataset. Once aggregated, the family-level biologic signature is stored in association with the user account and made available to one or more analytics engines for use in personalized health assessments.

The family-level biologic signature may be used as input to predictive or comparative analytics modules to derive insights into inherited disease risks, drug response likelihoods, donor compatibility for reproduction and fertility planning, and suitability for clinical trial inclusion. In certain embodiments, the biologic signature is compared to external reference datasets, including curated databases of hereditary conditions, population health benchmarks, and genomic risk profiles, to surface novel correlations and inform personalized medical recommendations. The recommendations may include, but not limited to, family and reproductive plannings, disease prevention strategies, clinical trial and personalized medicine recommendations.

The system supports a range of downstream applications for the biologic signature, including personalized treatment targeting, early disease screening strategies, and multi-generational health risk modeling. These insights can be exported, visualized, or embedded into user dashboards or external health platforms through the Data Presentation Layer (270), which supports interoperability with business intelligence tools and secure data formats. Privacy-preserving measures, such as de-identification, anonymization, and role-based access controls, ensure that the biologic signature and related analytics remain secure and compliant with applicable data protection laws.

Together, the system's data gap detection, enrichment, and biologic signature generation capabilities provide a comprehensive framework for capturing, completing, and leveraging family health data for high-impact, personalized medical and wellness applications. These features are tightly integrated with the system's permission management, executor designation, and analytics components to provide a legally compliant, biologically meaningful, and dynamically configurable health data ecosystem.

The following provides a more detailed discussion of the Health Estate Insights & Relationship (HEIR) System (100), as illustrated in FIG. 1. The system
architecture integrates multiple components for secure, permission-based health data access, management, filtering, analytics, and sharing across multiple family members and external data sources. FIG. 1 outlines the data flow, interactions between core system modules, and the role of each component in ensuring biologically and/or genetically relevant and legally compliant health data management.

The HEIR System (100) comprises interconnected modules that collectively facilitate the ingestion, storage, filtering, analysis, and controlled sharing of health data. Health Data Sources (115) provide structured patient health records, genetic testing results, and other medical data from hospitals, electronic health record (EHR) systems, insurance providers, and genetic laboratories. These data sources feed into an automated processing pipeline that cleans, standardizes, matches and structures the data before storing it in the Family Health Data Repository (130). In parallel, Reference & External Data Sources (190) contribute publicly available, institutional, or user-uploaded health datasets, such as clinical trial criteria, population benchmarks, and genetic reference libraries. These external datasets are separately processed and stored in the Reference Data Repository (200), ensuring that patient-specific records remain distinct from generalized reference datasets.

Data management within the HEIR System is structured around the Family Health Data Manager (140), which organizes health records for individual and connected family members. Each account within the system is assigned a unique PatientID (150), linking all associated health records from different data sources. The system further incorporates a Permission Management Service (120) that governs user consents, access rights, executor designations, and legal compliance for data sharing. The Graphical User Interface (GUI) (110) provides users with the ability to manage these permissions, facilitating family member connections, executor appointment and transfer, and controlled patient and family data sharing.

The Family Health Data Filtering Module (210) plays a critical role in ensuring that only biologically and/or genetically relevant and analytically meaningful data are included in system-based analyses. When a user initiates an analysis request, this module evaluates relationship metadata to classify family members by degrees of relatedness, distinguishing between biologically, genetically and non-biologically related individuals. The filtering module dynamically selects which health records to include based on the purpose of the analysis. For example, in cases where a user has a non-biologically related spouse but shares biological children with them, the system may include the spouse's data for offspring-related genetic risk assessments while excluding it from analyses focusing solely on the user's inherited conditions.

To address missing or incomplete health records, the HEIR System integrates a Family Health Data Gap Detection and Enrichment Service (230), as shown in FIG. 7. This module identifies data gaps, such as missing disease history, unreported diagnostic tests, or incomplete genomic markers, and applies machine learning techniques to estimate missing values. The enrichment process leverages a combination of predictive models, including a neural network trained on complete datasets from biologically and/or genetically related family members and an autoencoder trained to compress and reconstruct missing health data elements. These models are validated through a cross-validation module, ensuring that estimated values meet predefined confidence thresholds before they are integrated into the Family Health Data Repository (130). If confidence thresholds are not met or if inconsistencies arise, the system flags the estimated data for manual review, allowing the user or a designated health executor to verify its accuracy through the GUI (110).

A key feature of the HEIR System is its ability to generate a Family-Level Biologic Signature (260), as illustrated in FIG. 8. This biologic signature is a composite representation of biologic and health data aggregated across multiple connected family members, providing a more comprehensive view of inherited traits than traditional patient-level assessments. The biologic signature includes genetic sequences, protein markers, metabolites, biomarkers, immunologic profiles, and cellular pathway data, all of which are filtered through the Family Health Data Filtering Module (210) to ensure biological relevance. Once generated, the family-level biologic signature is stored in association with the user account and made available for downstream analysis.

The biologic signature serves as a foundation for predictive and comparative analytics, feeding into the system's Analytics Engine (220), which includes both Internal System Analytics Modules (240) and Third-Party Analytics Modules (250). These modules generate health risk scores, treatment compatibility assessments, donor compatibility evaluations, and clinical trial matching recommendations. The biologic signature may also be compared to external reference datasets stored in the Reference Data Repository (200) to identify correlations with known hereditary conditions, population health trends, and genetic predisposition models. The recommendations may include, but not limited to, family and reproductive plannings, disease prevention strategies, clinical trial and personalized medicine recommendations.

Once analytics are completed, results are processed through the Data Presentation Layer (270), which transforms outputs into various report formats, including dashboards, graphical visualizations, structured data exports, and machine-readable formats compatible with business intelligence tools such as Power BI and Tableau. This data can be accessed through the GUI (110), where users can configure reports, manage access permissions, and selectively share insights with approved family members, healthcare providers, or research institutions. The system enforces strict data security measures through the Communication Interface (280), which supports secure data transmission protocols, encryption mechanisms, and access controls that align with regulatory standards such as HIPAA, GDPR, and HITECH.

Through these interconnected modules, the HEIR System (100) enables a legally compliant, biologically and genetically meaningful, and dynamically configurable approach to intergenerational health data management. By integrating permission-based access control, biologically and genetically relevant data filtering, predictive analytics, and enrichment techniques, the system provides users with a robust platform for personalized healthcare, disease risk prediction, and family health planning. The combination of gap detection, family-based analytics, and biologic signature generation ensures that users have access to a comprehensive and continuously improving dataset that enhances medical decision-making and long-term health outcomes.

The following provides a more detailed discussion of the Family Member Connection & Verification Process (160) as illustrated in FIG. 2. This process ensures that users can establish authenticated connections with their family members within the system while maintaining data security, privacy, and integrity.

As shown in FIG. 2, the process begins when a user searches for a family member through the system's Graphical User Interface (GUI) (110). The system then determines whether the searched-for family member already has an account. If an account exists, the user may submit a connection request to link their account with the family member's profile. If no account is found, the user can send an invitation to the family member, prompting them to join the system and create an account. If the invited family member does not complete registration, the process terminates.

Once a connection request is submitted, the system prompts the recipient family member to either accept or decline the request. If the request is not accepted, the process ends. However, if the request is accepted, the system initiates an authentication procedure to verify the legitimacy of the connection. Both users are required to confirm the pending connection request, ensuring mutual recognition and preventing unauthorized or fraudulent linkages.

Each family member account involved in the request receives an authentication prompt instructing them to verify the connection's authenticity. The system then cross-validates the confirmation from both accounts. If either account does not verify the connection, the process terminates. If both users confirm the connection, the system formally establishes the family member link, and the verified connection is stored in the system's Family Health Data Repository (130) for future data sharing and analysis.

The Family Member Connection & Verification Process (160) ensures that all familial relationships recorded within the system are user-authenticated and verified, preventing unauthorized access to sensitive health data. By requiring mutual verification, the system strengthens security and enhances trust in intra-family health data sharing. This process also serves as a foundational step for permission-based access control, executor designation, and family health data filtering, enabling analytics that are biologically, genetically and legally relevant, within the HEIR System (100).

The following provides a more detailed discussion of the Executor Appointment and Transfer Process (170) as illustrated in FIG. 3. This process enables users to designate a Health Data Executor, who is granted legal permission to manage and access health data in the event of the original account owner's death or incapacity. This system ensures continuity of access to intergenerational health data while maintaining compliance with legal and privacy regulations.

The process begins when Family Member Account X initiates a request, through the system's Graphical User Interface (GUI) (110), to appoint another connected family member (Account Y) as their designated Health Data Executor. This request is sent electronically, and Family Member Account Y must accept the executor designation to proceed. If Account Y declines the request, the process terminates.

If Account Y accepts the request, both account holders are required to complete legal documentation within the system, formally recognizing the executor designation. This documentation may include HIPAA release forms, jurisdiction-specific consent agreements, and digital notarization features, ensuring compliance with federal and regional health data privacy and estate management laws. Once the documentation is completed, Family Member Account Y is officially assigned as the Health Data Executor for Family Member Account X.

In the event that Account X dies or becomes unable to manage their family health data, Account Y assumes full control over the account, gaining exclusive permissions to access, manage, and share the associated health records. This role grants Account Y the ability to facilitate health data access for estate settlement or generational health analysis.

To provide an additional layer of continuity, Family Member Account Y may also initiate a secondary executor designation by requesting another connected family member, Account Z, to serve as a backup Health Data Executor. This ensures that if Account

Y dies or becomes unable to fulfill their executor duties, Account Z can take over. If Account

Z accepts the request, the process continues, reinforcing long-term health data accessibility. If Account Z declines, the process terminates for the secondary executor designation, but Account Y remains the primary executor.

Over time, the Health Data Executor Transfer Process continues across multiple generations, enabling the seamless transfer of ownership and access permissions for ancestral health accounts to living descendants. This mechanism ensures that valuable health data remains available for medical reference, genetic analysis, and family health history tracking, even after the original data owners are deceased.

The Executor Appointment and Transfer Process (170) provides a secure, structured, and legally compliant method for transferring intergenerational health data, allowing families to maintain continuity of access and control over personal and hereditary health records. Through its multi-tier executor hierarchy, the system guarantees that health data remain accessible to designated successors while preventing unauthorized access, thereby preserving the integrity and privacy of the health data ecosystem.

The following provides a more detailed discussion of the HIPAA Release and Health Data Permission Management Interface, as illustrated in FIG. 4. This interface is part of the Health Data Management functionality within the system's Graphical User Interface (GUI) (110) and allows users to manage their health data sharing preferences, designate health data executors, and generate legally compliant HIPAA release forms.

At the top of FIG. 4, the user interface displays the PatientID (12345) and their account status, confirming the user's active status in the system. The GUI provides navigation options, allowing the user to access different sections, including Family Health History, Health Data Management, and Comparative Analytics, with the current screen focused on Health Data Management.

Within the Health Data Permissions Section, users are presented with multiple options, including appointing a health data executor, managing family member connections, and configuring family member data sharing preferences. This structured approach ensures that users can clearly define who has access to their health data and under what conditions.

A key element of this interface is the HIPAA Release Form Selection, which allows the user to generate a federal or state-specific HIPAA release document. In FIG. 4, the user has selected Massachusetts from the dropdown menu, indicating that the release form will comply with Massachusetts-specific privacy laws and regulations. The system dynamically loads the appropriate legal requirements based on the user's state selection.

The authorization section enables the user, identified as Patrick John Smith, to specify the extent of their health data disclosure. The user can opt to share their complete health record or selectively exclude specific categories of information, such as mental health records, communicable diseases, alcohol/drug abuse treatment records, genetic information, or other specified exclusions. In this case, the user has chosen to disclose their complete health record except for certain restricted categories.

Further down, the system allows the user to designate specific individuals or organizations with whom their health information will be shared. In FIG. 4, the user has entered the name Rebecca Todd-Smith as an authorized recipient, along with her address, confirming explicit authorization for data sharing with a designated individual.

The timeframe of authorization is also configurable. The user may select a fixed date range, allow access for all past, present, and future periods, or limit access until a specified event occurs. In this instance, the user has chosen to authorize access for all time periods, ensuring continued availability of health records to the designated recipient unless revoked.

To finalize the authorization, the user electronically signs the document by entering their name (Patrick John Smith), confirming the signature in the electronic signature field, and dating the authorization as Jan. 1, 2018. The system also includes a mechanism for revoking the authorization, ensuring compliance with HIPAA's requirement that individuals must be allowed to withdraw consent at any time.

Overall, FIG. 4 illustrates a legally compliant, user-friendly system for managing HIPAA authorizations, allowing users to securely and selectively share health data while maintaining control over their personal information. This module integrates with the Permission Management Service (120) to enforce access restrictions and ensure privacy compliance across different jurisdictions. By enabling state-specific HIPAA form generation, granular data-sharing preferences, and electronic consent validation, the system provides a comprehensive solution for managing health data permissions in a secure and legally enforceable manner.

The following provides a more detailed discussion of the Family Health Data Visualization and Risk Analysis Interface, as illustrated in FIG. 5. This interface is part of the Health Data & Executor Management module within the system's Graphical User Interface (GUI) (110) and is designed to provide users with a visual representation of family health relationships, health data executor assignments, and disease risk analytics.

At the top of FIG. 5, the interface displays Patrick John Smith's user profile, showing Patient ID: 23456 and an active status, confirming that the user is currently managing health data within the system. The interface also highlights Health Data Executor Appointments, listing Patient ID 12345 and Patient ID 44556, indicating that Patrick John Smith has been assigned as a health data executor for these family members.

On the right-hand side of FIG. 5, the Family Health Tree Visualization represents the user's family structure, showing relationships between the user and biologically, genetically or legally connected individuals. Each family member is represented by an icon, with identifiers for paternal and maternal grandparents, parents, uncles, aunts, and spouse. Notably, Patient ID 12345 (Mother) and Patient ID 44556 (Maternal Grandfather) are explicitly labeled, reinforcing that the system dynamically integrates and displays health data executor assignments alongside family connections.

The left panel of FIG. 5 presents Individual & Family Health Data, allowing users to navigate different categories of stored medical information. These categories include Patient Reported Data, Biologic Data, Clinical Data, Genetic Data, Imaging Data, Other Data, and Family Insights, reflecting the system's ability to consolidate diverse health datasets into an integrated repository.

A central feature of this interface is the Derived Disease Risk Score Analysis, which enables users to assess predicted health risks based on family health data. The user can select the scope of the family health analysis (e.g., individual, first-degree relatives, extended family, or all connected family members) and filter results by disease category. In FIG. 5, the disease category selected is cancer, and the system has generated risk scores for different cancer types, including breast cancer, colorectal cancer, endometrial cancer, kidney cancer, melanoma, and ovarian cancer. Each disease type is assigned a risk level (Low, Moderate, or High) based on a combination of genetic predisposition, family medical history, and biologic signature analysis.

A “Share” button is included in this section, allowing users to share their disease risk analysis with authorized family members, healthcare providers, or research institutions. This feature integrates with the system's Permission Management Service (120) to ensure that only designated individuals or organizations receive access to shared health insights.

The Family Health Data Visualization and Risk Analysis Interface in FIG. 5 demonstrates the system's ability to combine family health history data, health data executor management, and predictive analytics into a single, intuitive display. This enhances user decision-making, facilitates proactive health management, and enables informed genetic and medical risk assessments for both individual users and their extended family networks.

The following provides a more detailed discussion of the Family Member Account Data Sharing Process (180) as illustrated in FIG. 6. This process enables selective health data sharing between connected family members while maintaining granular control over data access permissions. It ensures that users can manage which portions of their health data are shared with specific family members, aligning with privacy preferences, legal compliance, and personalized health data needs.

FIG. 6 illustrates two distinct Patient IDs (150), representing Patient X and Patient Y, who are connected within the system. Patient X holds a broader dataset across multiple health data sources, while Patient Y receives only a subset of that data based on Patient X's sharing preferences. This structure demonstrates the controlled and customizable sharing of health data between authorized family members. At the bottom of FIG. 6, Patient X's full health dataset is depicted, organized by data sources. Patient X has health records from multiple sources, each containing several entries. Using the system's Graphical User Interface (GUI) (110) and Permission Management Module (120), Patient X selects a subset of health data to share with Patient Y, allowing for partial disclosure rather than granting full access to all available data. In the example illustrated, Patient X has chosen to share certain entries from different data sources while withholding others, demonstrating a highly configurable data-sharing mechanism.

This selective sharing mechanism ensures that Patient Y only receives a restricted and user-defined portion of Patient X's health records. The ability to filter and customize shared datasets allows for greater privacy control while still enabling meaningful health data exchanges between family members. The process is governed by the system's Family Member Connection & Verification Process (160) and enforced by role-based access permissions configured through the GUI (110). The system ensures that only authorized and verified family members can request and receive shared health data, that users have full autonomy over which data points are shared and can revoke access at any time, and that data-sharing preferences align with privacy regulations such as HIPAA or GDPR, ensuring compliance with legal standards.

The Family Member Account Data Sharing Process (180) enables controlled, privacy-preserving, and user-configurable data exchanges that facilitate intergenerational health insights, genetic analysis, and familial risk assessments while still maintaining data security and confidentiality. By allowing users to selectively share portions of their health data, the system provides a flexible and secure approach to managing family health records, ensuring that sensitive medical information is shared only as necessary and with appropriate safeguards in place.

The following provides a more detailed discussion of the Analytics Engine and Family Health Data Gap Detection & Enrichment Service, as illustrated in FIG. 7. This figure presents a comprehensive data analytics framework within the HEIR System (100), demonstrating how the system processes family health data, external reference datasets, and machine learning models to generate predictive insights, disease risk scores, and recommendations. The system leverages internal family insights (240), third-party insights (250), and automated enrichment algorithms to deliver a personalized, data-driven health intelligence platform. The recommendations may include, but not limited to, family and reproductive plannings, disease prevention strategies, clinical trial and personalized medicine recommendations.

The Analytics Engine (220) is the core computational module that processes health data from multiple sources, applies predictive models, and generates insights that may be applied to: clinical, scientific, family, legal and financial estate planning. The data ingestion process begins by integrating structured and unstructured health data from multiple sources, including but not limited to: electronic health records (EHRs), genetic testing results, self-reported family health histories, bio-imaging data, labs, immunizations and medication data, APIs, wearable sensors, medical devices, medical claims, patient charts and fertility data. These inputs are stored in the Family Health Data Repository (130) and processed for further analysis. Before any computation, the Permission Management Service (120) verifies that the data being accessed or shared complies with user-defined access permissions, legal consent settings, and privacy regulations such as HIPAA, GDPR, and other jurisdiction-specific policies.

A key function of the Analytics Engine (220) is to address missing or incomplete health data through the Family Health Data Gap Detection & Enrichment Service (230). This module employs machine learning algorithms to identify and estimate missing health information, ensuring that users have access to a complete and biologically relevant family health dataset. The data preprocessing phase cleans, normalizes, matches and structures health data before conducting gap detection analysis. The system then applies two primary machine learning models: (1) a Neural Network Estimation Model, which predicts missing health information based on patterns in family health data, and (2) an Autoencoder Reconstruction Model, which compresses existing health data into a latent space and reconstructs missing values based on learned correlations. These models work in conjunction with the Intelligent Family Health Data Filtering Module (210) to ensure that only biologically and/or genetically relevant data are used in predictive modeling, excluding non-biological family members where appropriate.

To validate estimated values, the system performs a cross-validation process, comparing outputs from multiple models and applying confidence thresholds to determine whether the inferred data meets reliability criteria. If the system achieves high-confidence estimations, the new data is integrated into the Family Health Dataset (130). If discrepancies arise, the system flags the data for manual review via the GUI (110), where users or designated Health Data Executors can either approve or reject the enriched data before it becomes part of the official health record.

The system's Internal Analytics Module(s) (240) processes biologic signatures, genetic predispositions, and familial disease trends to provide actionable insights based on direct family history. This module consolidates inherited health risks, identifies common disease patterns within a user's family network, and supports personalized medicine recommendations based on aggregated genetic, clinical, and environmental data. The system further enhances its predictive capabilities through the Third-Party Insights Module (250), which integrates external reference datasets, clinical trial inclusion criteria, population health data, and research-based risk assessment models. These third-party sources augment the system's predictive analytics, ensuring that family health data is contextualized within broader medical and epidemiological frameworks. The recommendations may include, but not limited to, family and reproductive plannings, disease prevention strategies, clinical trial and personalized medicine recommendations.

The system's Analytics Engine (220) generates personalized disease risk scores for various conditions, including cardiovascular diseases, diabetes, cancer, neurodegenerative disorders, autoimmune diseases, and rare genetic conditions. These scores are derived from both internal family data and external third-party data, creating a hybrid risk assessment model that is more accurate and comprehensive than individual genetic testing alone. Based on these insights, the system automatically generates health recommendations, which may include preventive screening schedules, lifestyle modification suggestions, genetic counseling referrals, and personalized treatment options.

To make these analytics accessible, the system outputs results through the Data Presentation Layer (270), which formats health insights into interactive reports, visualizations, and structured datasets. Users can access their personalized health risk profile, family disease trends, and biologic signature insights via the Graphical User Interface (GUI) (110) or export their reports to external platforms, including business intelligence tools, healthcare applications, and secure data-sharing networks. The Communication Interface (280) enables users to securely share their insights with designated family members, physicians, or research institutions, ensuring that only authorized recipients can view or act upon the shared data.

FIG. 7 illustrates a sophisticated and highly structured analytics architecture, where family health data, external medical knowledge, and machine learning-driven enrichment work together to deliver accurate, predictive, and biologically relevant health intelligence. The integration of Family Insights (240), Third-Party Insights (250), and the Family Health Data Gap Detection & Enrichment Service (230) ensures that users receive a holistic, data-driven understanding of their genetic, biologic and medical risk factors. This system not only enhances personalized healthcare planning but also facilitates intergenerational health insights, enabling families to make informed decisions regarding preventive care, genetic screening, and long-term medical management.

The following provides a more detailed discussion of the Family Biologic Signature (260) as illustrated in FIG. 8. This figure presents a composite health data framework that consolidates genetic, proteomic, epigenetic, metabolic, physiologic, and lifestyle data from multiple family members to generate a biologically and/or genetically relevant signature at the family level. The Family Biologic Signature is used for predictive analytics, disease risk assessments, personalized medicine, and multi-generational health monitoring, and more.

FIG. 8 contains a structured dataset where each row corresponds to a unique Patient ID, representing different family members in a user's verified family network. The columns encompass a broad spectrum of biologic data categories, including gene mutations, single nucleotide polymorphisms (SNPs), epigenetic markers, metabolite biomarkers, transcriptomic activity, and physiological indicators. The system may use a binary detection system (1 or 0) for genetic and molecular features, where a value of “1” denotes the presence of a biomarker, mutation, or detectable epigenetic modification, while a value of “0” indicates the absence of such a feature, for example. However, other suitable measurement systems may also be employed.

The dataset integrates several key biological parameters. The Medical & Lifestyle Data Derived from Patient EHR, Claims, etc. column aggregates clinically recorded
health behaviors, environmental exposures, and chronic disease history. The Single Nucleotide Polymorphisms (SNPs) and Gene Mutations column detects inherited genetic variations that may predispose individuals to conditions such as cancer, cardiovascular diseases, or metabolic disorders. The Epigenetic & Transcriptomic Data (Gene Expression) column measures gene expression levels, where heat map shading differentiates upregulated (red) and downregulated (green) genes, with black indicating no significant expression change. Metabolite and Microbiome Data quantify circulating biomarkers, including proteins, amino acids, and metabolites, that reflect an individual's metabolic and microbiome composition.

To establish biologic relevance, the system may incorporate correlation analyses using Pearson Correlation Coefficients, which assess statistical relationships between continuous variables such as gene expression, protein levels, and disease risk. Strong correlations (values above 0.6) suggest biologically significant associations between genetic factors and disease phenotypes, aiding in personalized risk assessments and treatment stratification. Additionally, the DNA Methylation Profile measures the percentage of methylated regions within normal or tumor tissue, providing insights into cancer risk and epigenetic modifications that affect gene regulation. It should be noted that other suitable measurement systems can also be employed.

The Family Biologic Signature (260) provides a holistic representation of inherited health risks, allowing the system to generate predictive models for disease susceptibility, identify shared familial health trends, and recommend personalized treatment options. By consolidating multi-omic data (genetic, epigenetic, transcriptomic, metabolomic, and physiologic indicators) across family members, the system enhances its predictive power beyond traditional single-patient analysis. The biologic signature framework also enables comparative analytics, allowing users to benchmark their family's health profile against population health databases, clinical trial eligibility criteria, and personalized medicine repositories.

FIG. 8 illustrates a highly structured, data-driven approach to family health analytics, supporting personalized healthcare, disease prevention, and multi-generational health planning. Through its integration with the Analytics Engine (220), Family Insights Module (240), and Third-Party Insights Module (250), the Family Biologic Signature serves as a foundational dataset for real-time monitoring, automated risk assessment, and precision medicine applications.

The following provides a more detailed discussion of FIG. 9, which illustrates different methods of comparative health data analysis enabled by the system. FIG. 9 consists of three sub-FIGS. 9a, 9b, and 9c, each demonstrating a different approach for comparing an individual's family health data against another individual, multiple clusters of family health datasets, or reference datasets from external sources. These comparative analysis methods are facilitated by the Permission Management Service (120), Family Health Data Repository (130), and Reference Data Repository (200).

The system of the present disclosure, as illustrated in FIG. 9, enables various comparative health data analysis methods by allowing users to benchmark their individual and family health data against other users, multiple family clusters, or external datasets. The Permission Management Service (120), Family Health Data Repository (130), and Reference Data Repository (200) work together to facilitate secure, consent-based comparative analytics while ensuring compliance with privacy regulations and data security protocols.

As illustrated in FIG. 9a, the system supports direct comparative analysis between two distinct Patient IDs (150), represented as Patient ID A1 and Patient ID B1. Each Patient ID corresponds to a multi-generational family network, consisting of genetic, clinical, and biologic health data. Before conducting a comparative analysis, the Permission Management Service (120) ensures that both users have granted explicit consent to share their family health data. This method allows two users to compare individual and family health data.

The system of the present disclosure, as illustrated in FIG. 9b, expands this functionality to allow comparative analysis across multiple clusters of family health datasets. In this scenario, Patient ID A1 can analyze their family health data alongside several other family clusters, such as Patient ID B1, C1, D1, and E1. Each cluster represents a separate family unit with distinct genetic markers, disease risks, and biologic profiles. This approach enables large-scale comparative epidemiological studies, supporting insights into shared hereditary conditions, treatment outcomes, and multi-family disease modeling. The Permission Management Service (120) ensures that only authorized family clusters participate, preserving data confidentiality and access controls.

The system of the present disclosure, as illustrated in FIG. 9c, enables comparative benchmarking between a family health dataset and external medical reference datasets (190) stored in the Reference Data Repository (200). In this method, Patient ID A1 can compare their family health data against curated datasets from clinical trials, genomic research, public health benchmarks, and population-wide epidemiological studies. This functionality allows users to assess their genetic risk factors in the context of broader scientific findings, improving disease risk prediction and treatment personalization. Users can either manually select specific reference datasets or allow the system to automatically recommend the most relevant datasets based on their family health profile and biologic signature.

The system of the present disclosure enables advanced comparative health analytics, allowing users to gain deep insights into their hereditary risk factors, treatment compatibilities, and overall health standing relative to broader datasets. The Permission Management Service (120), Family Health Data Filtering Module (210), and Analytics Engine (220) work together to ensure that all comparative analyses are secure, relevant, and compliant with regulatory standards. Through these comparative models, the system supports personalized medicine, early disease detection, and multi-generational health planning, offering users an advanced platform for data-driven healthcare decision-making.

The system of the present disclosure, as illustrated in FIG. 10, enables comparative analytics for family health insights, fertility planning, disease risk assessment, and personalized medicine. This interface provides users with a visual and data-driven approach to assessing hereditary disease risks and evaluating compatibility with external health datasets, such as fertility and genomic reference profiles.

At the top of the interface, the Comparative Analytics Module presents multiple options, including Clinical Trial Opportunities, Family and Fertility Planning, Personalized Medicine Insights, and Disease Prevention and Screening Insights. The active selection in this example is Family and Fertility Planning, indicating that the user, Janet Jones (Patient ID: 43452), is comparing her family health data against a fertility reference dataset (Fertility Library XXZ) and a specific comparator profile (Profile XYZ). The system integrates information from the Family Health Data Repository (130) and Reference Data Repository (200) to enable real-time comparisons between a user's individual and/or family health data and external datasets and profiles.

On the left side of the interface, the system displays Janet Jones'family health data, illustrating a family tree with first-and second-degree relatives. The Comparator Data Set on the right shows Profile XYZ, a prospective reference individual, including attributes such as gender (male) and date of birth (DOB: XX/XX/XXXX). The Permission Management Service (120) ensures that data-sharing permissions are in place before conducting any comparative analysis.

Below the family tree visualization, the Disease & Hereditary Condition Risk Assessment Module enables users to assess disease risks for progeny based on family medical history, biologic and genetic data. The user has selected Cancer as the category of interest and restricted the analysis to first-and second-degree familial connections. The system has generated a calculated risk assessment for various hereditary cancer conditions, including BRCA1 and BRCA2 mutations, colorectal cancer, familial adenomatous polyposis (FAP), Lynch syndrome, and prostate cancer. The risk levels for each condition range from low to moderate, offering insights into potential hereditary predispositions for offspring. Conditions marked with an asterisk (*) indicate well-established hereditary risk factors linked to increased cancer susceptibility.

The system of the present disclosure integrates predictive analytics and risk modeling techniques within the Analytics Engine (220) to generate risk assessments. The system dynamically evaluates genetic predispositions, family history, and comparator profile attributes, providing actionable insights for fertility planning, genetic counseling, and medical decision-making. The Data Presentation Layer (270) enables users to share, export, or further analyze the generated insights in real-time health applications, genetic counseling sessions, or clinical consultations.

Through the Comparative Analytics Module, Disease Risk Assessment Tools, and Reference Data Benchmarking, the system provides a comprehensive and privacy-compliant platform for evaluating hereditary disease risks, fertility compatibility, and predictive health modeling. By integrating user family health data, external genomic datasets, and real-time analytics, the system enables personalized decision-making for reproductive planning, disease prevention, and proactive healthcare management.

The system of the present disclosure, as illustrated in FIG. 11, provides a Family Health & Biosignature Data Configuration Tool that enables users to configure, analyze, and export multi-dimensional health data from individuals, families, and broader population datasets. This interface is designed to facilitate secure, privacy-compliant data selection, analysis, de-identification, and sharing through the system's Data Presentation Layer (270), Communication Interface (280), and Data Sharing Module (290).

At the top of the interface, users can select export parameters based on their needs. The system allows users to retrieve Family Health & Biosignature Data for either a specific connected family network (a group of linked Patient IDs) or a batch of families from the system database. Users can search for unique Patient IDs and configure batch settings based on demographic filters, enabling targeted data extraction. The scope of family health data can be configured to include first-and second-degree relatives only or an expanded set of extended family members. Additionally, users can define a date range for the data export, selecting between a specific time frame or all available data.

The system provides a highly configurable data inclusion module, allowing users to select which types of health data are retrieved for export. Available data categories include clinical data (EHR, claims), epigenetic data (DNA methylation, histone modifications), genomic and genetic data (gene markers, SNPs), imaging data (CT scans, MRI), lifestyle and patient-reported data, microbiome data, physiological data (wearables, sensors), proteomics data (protein expression, metabolite markers), and transcriptomic data (mRNA expression, cytokine activity). Users can also determine whether an analytics layer is applied to the exported dataset, providing real-time insights or raw data extraction without additional processing.

To ensure privacy and compliance with data protection laws, the system includes an Anonymization & De-Identification Module that allows users to remove PHI (Protected Health Information) before exporting. Users can select the output file format, such as PDF, CSV, or JSON, depending on their intended use case.

The Data Presentation Layer (270) plays a key role in structuring and securing data before export. This layer is responsible for data selection, de-identification, identity management, role-based access control (RBAC), attribute-based access control (ABAC), and encryption protocols to ensure compliance with industry regulations such as HIPAA, GDPR, and HITECH. The system ensures that all exported data maintain confidentiality and security while remaining interoperable with external platforms.

Below the configuration tool, FIG. 11 displays a structured dataset representing multi-source family health and biosignature data, with individual Patient IDs mapped to biologic, genetic, and environmental health markers. The table includes gene mutations, SNPs, transcriptomic and proteomic data, physiologic parameters, and correlation-based risk scores for disease prediction and personalized medicine applications. The structured format enables researchers, clinicians, and authorized family members to extract valuable insights for disease risk modeling, treatment optimization, and familial health tracking.

The Communication Interface (280) and Data Sharing Module (290) ensure that data can be securely transmitted to authorized recipients, external platforms, and healthcare providers. The system supports multiple data-sharing methods, including Secure FTP (SFTP) for bulk file transfers, API-based integrations for real-time interoperability, and direct EHR/EMR system exports for seamless clinical data exchange. The encryption and access control mechanisms ensure that only authorized personnel can access shared datasets, preventing unauthorized use or data breaches.

The system of the present disclosure, as illustrated in FIG. 11, enables secure, configurable, and privacy-compliant extraction of family health and biosignature data for clinical research, comparative analytics, and multi-generational health tracking. By incorporating advanced data selection, analysis, anonymization, and interoperability features, the system facilitates precision medicine, predictive analytics, and secure health data sharing in a scalable and compliant framework.

The system of the present disclosure provides a multi-layered, automated, and privacy-compliant platform for managing, processing, and analyzing multi-generational family health data, with specific applications in disease risk prediction, biologic signature analysis, health data permissioning, and secure data exchange across different healthcare and research environments. Unlike conventional electronic health record (EHR) systems or static family history databases, the present system introduces real-time data enrichment, Filtering for biologic, genetic and legal relevance, and a structured, role-based permission model that enables hierarchical access control over sensitive family health information.

Technical Improvements Over Conventional Systems.

Existing health data management systems lack intelligent, biologically and genetically relevant filtering of consolidated and linked family health data, resulting in incomplete or inaccurate assessments of familial disease risk. Additionally, these systems do not dynamically identify or reconstruct missing health data points based on machine learning-based inference models. The present system addresses these challenges by introducing a novel computational architecture that ensures complete, accurate, and privacy-protected family health insights.

At the core of this architecture is the Analytics Engine (220), which applies machine learning-driven data enrichment techniques through the Family Health Data Gap Detection & Enrichment Service (230). This service employs a multi-phase process to detect and reconstruct missing health data points. The system first preprocesses health data by cleansing, normalizing, matching and validating existing records stored in the Family Health Data Repository (130). If gaps in patient or family health histories are detected, the system applies a trained neural network model to estimate missing values based on familial, genetic, and biologic correlations.

In contrast to conventional static imputation models, the present system incorporates an autoencoder-based reconstruction method that maps existing health data points into a compressed latent space and generates highly accurate estimations for missing attributes. The estimated data is then cross-validated using a hybrid statistical inference model to ensure high-confidence enrichment, after which the updated health record is stored and made available through the Graphical User Interface (GUI) (110). If the system detects discrepancies between models, it flags the enriched data for manual validation by an authorized user.

Hierarchical, Multi-Tier Permission-Based Health Data Sharing.

A key technological advantage of the system is its advanced hierarchical permissioning model, implemented via the Permission Management Service (120). Unlike conventional EHRs that provide binary (full or no access) patient data permissions, the present system enables granular access control over multi-generational health data. Through the Health Data Executor Appointment and Transfer Process (170), users can designate, modify, or revoke access permissions based on life events, legal authorizations (e.g., HIPAA consents), and role-based inheritance mechanisms.

The system ensures that health data access follows a structured, event-driven permissioning hierarchy. For example, upon the passing of a primary account holder, the designated Health Data Executor automatically inherits tiered access to stored medical records, ensuring that familial health history data remains accessible for future generations while maintaining legal and privacy compliance. Additionally, the system implements real-time permission validation mechanisms, wherein the Permission Management Service (120) interacts with the Communication Interface (280) to ensure that shared data complies with role-based access control (RBAC), attribute-based access control (ABAC), and industry regulations (e.g., HIPAA, GDPR, HITECH).

Intelligent Family Health Data Filtering.

To ensure that only biologically and/or genetically relevant health data are included in analysis, the system implements an Intelligent Family Health Data Filtering Module (210). This module applies dynamic filtering mechanisms to:

Identify biologically and/or genetically related family members and exclude all or some of an individual's genetic, biologic and/or non-biological family members (e.g., in-laws, step-relations) unless explicitly included.

Allow contextual filtering of family health data for different analytical purposes (e.g., excluding paternal-side relatives in maternal-line disease assessments).

Provide automated scope adjustment for multi-generational health trend analysis, ensuring dynamic adaptation of data filtering rules based on the specific analytic request.

Unlike conventional data selection mechanisms, which require manual input from users, the present system automates biologic relevance filtering based on genetic, epidemiological, and familial health patterns, ensuring that predictive models operate with higher accuracy and efficiency.

Real-Time, Privacy-Compliant Health Data Sharing.

The system integrates a multi-layered security and encryption framework via the Communication Interface (280) and Data Sharing Module (290). Conventional health data exchange mechanisms, such as EHR-based patient portals, lack fine-grained privacy controls and typically rely on manual consent processes. In contrast, the present system implements automated encryption, role-based authentication, and audit logging mechanisms that ensure secure and compliant data transmission.

The system enables multiple modes of secure data transfer, including:

Secure API-based integrations for interoperability with third-party platforms.

Encrypted file transfer protocols (SFTP, FTPS, HTTPS) to facilitate research collaborations.

De-identified data sharing, ensuring that personal identifiers are removed before data transmission.

Customizable access windows, allowing users to define time-limited data-sharing permissions (e.g., granting access to a medical professional for a predefined period before auto-revocation).

These features ensure that the system is not merely a generic health data aggregation tool, but a technically robust, privacy-aware, and legally compliant solution for handling multi-generational, biologically and/or genetically relevant health records.

In accordance with some embodiments, the system of the present disclosure, as illustrated in FIG. 1 and related figures, includes a platform for managing intergenerational health data access and control. The system includes a data repository (130) configured to store health data associated with a plurality of family members. This health data includes electronic health records, genetic testing results, biomarkers, bio-imaging, and self-reported health information, to name a few. In communication with the data repository is a permission management module (120), which is configured to solicit, receive, centralize, and store access permissions and legal consent documentation, including but not limited to Health Insurance Portability and Accountability Act (HIPAA) authorizations, that govern access to the health data of one or more family members. The permission management module also enables users to designate one or more individuals as health data executors and facilitates the transfer of access rights to such executors or other designated family members in response to predefined triggering events, such as user incapacity or death. In addition, the module enforces jurisdiction-specific legal, privacy, and data access requirements based on the geographic region associated with either the data subject or the requesting party. The system further includes a communication module and the data repository, the interface being configured to interact with external data systems—such as healthcare provider systems and insurance databases—to retrieve, update, and manage health data in accordance with the stored permissions and executor designations.

In some embodiments, the system further includes a family health data filtering module (210), as shown in FIG. 7, configured to identify and categorize family member relationships associated with each user account, including first-, second-, and third-degree relatives. The filtering module is also capable of distinguishing biologically and genetically related family members from non-biologically related members based on stored relationship metadata or user input. Additionally, the filtering module is configured to dynamically include or exclude the health data of certain family members from system-based analyses depending on the nature of the analysis, such as individual risk prediction versus multi-generational trend modeling. In specific scenarios, the module permits the selective inclusion of health data from a non-biologically related family member who shares biological children with a user, allowing for the inclusion of that data in offspring-related analytics.

The system further enhances its filtering functionality by utilizing clustering algorithms or relationship graph analysis to generate biologically and/or genetically relevant subsets of family members tailored to the analytic task. It also accepts user-defined rules, entered via the GUI (110) depicted in FIG. 11, that customize which family connections and associated health data are included in particular analyses or data visualizations. In some implementations, the family health data filtering module applies filtering parameters that adapt based on jurisdictional definitions of family or legal relationships, ensuring compliance with local regulations. Moreover, the filtering module interfaces directly with the permission management module to ensure that only data from authorized and verified family member connections are included in any given analysis.

The present disclosure also encompasses a computer-implemented method for managing intergenerational access to health data. This method includes storing, in a data repository (130), health data for a plurality of family members, including electronic health records, biologic and genetic testing data, and self-reported health information. Via a graphical user interface (110), the system receives one or more legal consent documents authorizing access to health data. These consent documents may include executor designations and jurisdiction-specific privacy settings. The system stores the received permissions in the permission interface (280) operatively connected to both the permission management management module (120) and associates them with respective health data records in the repository. Upon detecting a triggering event associated with a user account, such as user incapacity or death, the system transfers access rights for the affected user's health data to a designated health data executor. Simultaneously, the system retrieves or updates health data from one or more external systems through the communication interface (280), ensuring that all data interactions are governed by the permissions stored within the permission management module.

This method also includes identifying relationships between a user and a plurality of connected family members, including first-, second-, and third-degree relatives. The system distinguishes, using the family health data filtering module (210), between biologically, genetically and non-biologically related family members based on stored metadata, user input, or both. The method enables dynamic selection of health data associated with biologically and/or genetically related family members for use in predictive, comparative, prescriptive or descriptive analytics processes. In cases involving shared offspring, the system allows for the inclusion of health data from non-biologically related family members in offspring-related analytics, while excluding such data from analyses unrelated to the shared children. Data clustering techniques or relationship graph analysis may be applied to generate a biologically and/or genetically relevant family subset for system-generated analytics. Additionally, users may input custom filtering rules through the GUI, which determine which family connections and health data should be included in a given analysis or report.

The system also filters family member data based on legal or jurisdictional definitions of permitted relationships and data access rights. Before executing any analytic operation, the system verifies—through the permission management module—that only data from authorized and verified family member accounts are included in the analysis.

Further, the present disclosure includes a computer-implemented method for filtering family health data for analysis. This method involves identifying, by a computing system, a plurality of family member accounts associated with a user account, each account comprising health data and relationship metadata. The system determines, based on that metadata, the degree of relatedness between the user and the family member accounts, including at least whether the relationships are biologically, genetically or non-biologically related. Upon receiving, through the GUI (110), a request to perform health data analysis with one or more analysis parameters, the family health data filtering module (210) applies one or more filtering rules to selectively include or exclude health data from the associated family accounts based on the degree of relatedness and the selected analysis type. Based on this filtered family health data, the system generates a dataset for input into the analytics engine (220) for use in health and biological risk assessments, predictive modeling, or comparative analysis. In certain configurations, the filtering rules include a provision to include health data from a non-biologically or genetically related family member in the analysis if that person shares one or more biological children with the user account, while excluding that same family member's data from analyses unrelated to the shared offspring.

In accordance with some embodiments, the system of the present disclosure, as illustrated in FIG. 7 and related figures, includes a system for performing health data gap detection and enrichment across family health records. The system includes a data repository (130) configured to store health data associated with a plurality of user accounts and corresponding family member accounts. The stored health data includes electronic health records, genetic data, biologic data, and self-reported information. A gap detection module is configured to identify missing or incomplete health data associated with a user account and one or more connected family member accounts. An enrichment engine, in communication with the gap detection module, is configured to estimate missing health data using one or more machine learning models trained on verified family health data, the models including at least a neural network and an autoencoder. A cross-validation module is further configured to compare outputs from the machine learning models and apply confidence thresholds to validate estimated values. A data integration module is configured to incorporate validated estimated data into the user's family health dataset stored in the data repository (130), or to flag unvalidated estimates for manual review via a graphical user interface (110).

In some embodiments, the system further includes a family health data filtering module (210) configured to select a biologically and/or genetically relevant subset of family members for inclusion in the gap analysis and estimation process based on stored relationship metadata. The system may also include a preprocessing module that cleans, normalizes, matches and structures incoming health data prior to gap detection, ensuring compatibility with the machine learning-based enrichment process. The enrichment engine may use a neural network trained on complete datasets from biologically and/or genetically related family member accounts to infer missing values based on genetic, medical, biologic, and familial patterns. Alternatively, or in addition, the enrichment engine may apply an autoencoder to encode known health data into a latent representation and reconstruct missing data elements based on pattern learning.

The system may further include a biologic signature generation module (as shown in FIG. 8) configured to consolidate biologic, genetic and health data across multiple connected PatientIDs to generate a family-level biologic signature. The biologic data used to generate the signature includes, but is not limited to, genes, proteins, metabolites, biomarkers, and cellular activity indicators. The family-level biologic signature may then be used as input to one or more analytics modules (240 and 250 in FIG. 7) to generate predictive, prescriptive, descriptive or comparative health insights, including but not limited to personalized treatment recommendations, disease risk assessments, and clinical trial matching suggestions.

The present disclosure also includes a computer-implemented method for detecting and enriching health data gaps in a family health dataset. This method includes storing, in a data repository, health data associated with a user account and a plurality of connected family member accounts, with the health data including clinical, genetic, and biologic information. The system identifies, via a gap detection module, one or more missing or incomplete data elements associated with the user account or the user's broader family health dataset. It then applies one or more machine learning models to estimate the missing data elements, including at least a neural network and an autoencoder, each trained on verified family health data from biologically and/or genetically related accounts. The system compares the outputs of the machine learning models using a cross-validation module to determine whether the estimated values satisfy a predefined confidence threshold. The estimated values that meet this threshold are integrated into the family health dataset, while others are flagged for manual review via the graphical user interface (110).

In certain embodiments, the method further includes preprocessing the stored health data by cleaning, normalizing, matching and structuring the data for compatibility with the gap detection and enrichment pipeline. The system may also filter the user's connected family member accounts to identify only biologically and/or genetically related individuals for inclusion in the estimation process, using the family health data filtering module (210). In the neural network implementation, missing data are estimated based on identified familial, genetic, and biologic correlations from related accounts. In the autoencoder implementation, existing health data are encoded into a latent vector and used to reconstruct missing data points based on learned representations.

The system may also generate a family-level biologic signature by aggregating biologic data across multiple family member accounts. The biologic data may include gene expression data, protein markers, cellular pathway information, and metabolite profiles. Once generated, this family-level biologic signature can be used as input for the system's analytics engine (220), which in turn can provide personalized medicine recommendations, disease risk scores, and compatibility assessments for fertility or donor selection, as shown in FIG. 10.

The system further includes a computer-implemented method for generating a family-level biologic signature. This method includes identifying, via a computing system, a user account and a plurality of connected family member accounts, each associated with respective biologic, genetic and health data. The system retrieves biologic data associated with the user account and the connected family member accounts from the data repository, with the biologic data including genetic information, protein markers, metabolites, and biomarkers. The retrieved data are filtered to include only data from family member accounts that meet predefined biological, genetic or relational relevance criteria. The system aggregates the filtered biologic data to generate a consolidated family-level biologic signature that characterizes inherited and familial biologic traits, and stores this signature in association with the user account for use in downstream analysis.

In some embodiments, the biologic data further include gene expression data, proteomics, metabolic pathways, cell signaling pathways, immunologic markers, and image-based biologic features such as radiographic or imaging biomarkers. The relational relevance criteria for including family member data may comprise first-, second-, or third-degree biologically and/or genetically related family members, as determined by relationship metadata stored in the system. Based on the generated family-level biologic signature, the system may produce health insights including disease risk predictions, treatment effectiveness estimates, or donor compatibility assessments.

The family-level biologic signature may also serve as input to a predictive analytics engine configured to identify personalized medicine opportunities or clinical trial matches for the user account, using internal analytics modules (240) or third-party analytics services (250). Additionally, the system may compare the generated biologic signature to one or more external reference datasets stored in the reference data repository (200), in order to identify correlations with known hereditary conditions or population-level health benchmarks. These comparative analyses, as shown in FIG. 9 and FIG. 10, enable users to contextualize their family biologic, genetic and health profiles relative to clinical data libraries, diagnostic thresholds, and eligibility criteria for targeted interventions.

The system of the present disclosure represents a significant technological advancement over conventional health data storage and sharing platforms by introducing automated family health data enrichment, biologically and genetically relevant filtering, and hierarchical permission-based data access models. Through the Family Biologic Signature (260), Intelligent Family Health Data Filtering Module (210), and Machine Learning-Based Data Gap Enrichment Service (230), the system ensures completeness, accuracy, and contextual relevance of family health insights, enabling precision medicine, predictive analytics, and intergenerational health planning.

By implementing real-time encryption, structured access controls, and compliance with local and global health data security regulations, the system ensures that sensitive medical records remain protected while providing users with an advanced tool for personalized healthcare management. Unlike conventional systems that rely on manual data entry, rigid access control models, or static rule-based analytics, the present system introduces a dynamic, learning-based approach to multi-generational health insights, making it a novel and patent-eligible technological improvement.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

Although the invention is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements.

The foregoing detailed description is merely exemplary in nature and is not intended to limit the invention or application and uses of the invention. Furthermore, there is no intention to be bound by any expressed or implied theory presented in the preceding technical field, background, brief summary, or the following detailed description.

What is claimed is:

1. A system for performing health data gap detection and enrichment across family health records, comprising:
a data repository configured to store health data associated with a plurality of user accounts and corresponding family member accounts, the health data comprising electronic health records, genetic data, biologic data, and self-reported information;
a gap detection module configured to identify missing or incomplete health data associated with a user account and one or more connected family member accounts;
an enrichment engine in communication with the gap detection module, the enrichment engine configured to estimate missing health data using one or more machine learning models trained on verified family health data including relatives and non-relatives and third-party data sets, the models comprising at least a neural network and an autoencoder;
a cross-validation module configured to compare outputs from the machine learning models and apply confidence thresholds to validate estimated values; and
a data integration module configured to incorporate validated estimated data into the user's family health dataset stored in the data repository, or flag unvalidated estimates for manual review via a graphical user interface.

2. The system of claim 1, further comprising a family health data filtering module configured to select a biologically and/or genetically relevant subset of family members for inclusion in the gap analysis and estimation process based on relationship metadata.

3. The system of claim 1, further comprising a preprocessing module configured to clean, normalize, match and structure incoming health data prior to gap detection.

4. The system of claim 1, wherein the enrichment engine uses one or more machine learning models, including a neural network trained on complete datasets from verified family member accounts and/or external datasets, to infer missing values based on genetic, biologic medical, and familial patterns.

5. The system of claim 1, wherein the enrichment engine uses an autoencoder to encode known health data into a latent representation and reconstruct missing data elements based on pattern learning.

6. The system of claim 1, further comprising a biologic signature generation module configured to consolidate biologic and health data across multiple connected PatientIDs to generate a Family-Level Biologic Signature, the biologic data comprising genes, proteins, metabolites, biomarkers, bio-imaging and cellular activity indicators.

7. The system of claim 6, wherein the Family-Level Biologic Signature is used as input to one or more analytics modules to generate predictive, descriptive, prescriptive or comparative health insights, including personalized treatment recommendations, disease risk assessments, fertility and family planning recommendations or clinical trial matching.

8. A computer-implemented method for detecting and enriching health data gaps in a family health dataset, comprising:
storing, in a data repository, health data associated with a user account and a plurality of connected family member accounts, the health data comprising at least clinical, genetic, and biologic information;
identifying, by a gap detection module, one or more missing or incomplete data elements associated with the user account or the user's family health dataset;
applying one or more machine learning models to estimate the missing data elements, the models comprising at least a neural network and an autoencoder, each trained on verified family health data from biologically and/or genetically related accounts; comparing outputs of the machine learning models using a cross-validation module to determine whether the estimated values satisfy a predefined confidence threshold;
and integrating the estimated values that meet the confidence threshold into the family health dataset, or flagging the values for manual review via a graphical user interface.

9. The method of claim 8, further comprising preprocessing the stored health data by cleaning, normalizing, matching and transforming the data for compatibility with the gap detection module and enrichment engine.

10. The method of claim 8, further comprising filtering the user's connected family member accounts to identify only biologically and/or genetically related individuals for inclusion in the estimation process.

11. The method of claim 8, wherein the neural network estimates missing data based on identified familial, genetic, and biologic correlations from related accounts.

12. The method of claim 8, wherein the autoencoder encodes existing health data into a latent vector and reconstructs missing data points based on learned patterns.

13. The method of claim 8, further comprising generating a Family-Level Biologic Signature by aggregating biologic data across multiple family member accounts, the biologic data comprising at least gene expression data, protein markers, cellular pathway information, bio-imaging and metabolite profiles.

14. The method of claim 13, further comprising using the Family-Level Biologic Signature as input for an analytics engine to generate personalized medicine recommendations, disease risk scores, or compatibility assessments for fertility or donor selection.

15. A computer-implemented method for generating a family-level biologic signature, comprising:
identifying, by a computing system, a user account and a plurality of connected family member accounts, each associated with respective biologic and health data;
retrieving, from a data repository, biologic, genetic and medical data associated with the user account and the connected family member accounts, the biologic, genetic and medical data comprising at least genetic information, protein markers, metabolites, bio-imaging and biomarkers;
filtering the retrieved biologic data to include only data from family member accounts that meet predefined biological or relational relevance criteria;
aggregating the filtered biologic, genetic and medical data to generate a consolidated family-level biologic signature that characterizes inherited and familial biologic and genetic traits; and storing the family-level biologic signature in association with the user account for use in downstream analysis.

16. The method of claim 15, wherein the biologic data further comprise data related to gene expression, proteomics, metabolic pathways, cell signaling pathways, immunologic markers, and image-based biologic features.

17. The method of claim 15, wherein the relational relevance criteria comprise inclusion of first-, second-, or third-degree biologically and/or genetically related family members based on relationship metadata.

18. The method of claim 15, further comprising generating one or more health insights based on the family-level biologic signature, the insights comprising disease risk predictions, treatment effectiveness estimates, or fertility donor compatibility assessments.

19. The method of claim 15, wherein the family-level biologic signature is used as input to a predictive analytics engine configured to identify personalized medicine opportunities or clinical trial matches for the user account and provide fertility and family planning recommendations.

20. The method of claim 15, further comprising comparing the family-level biologic signature to one or more external reference datasets to identify correlations with known hereditary conditions or population-level health benchmarks.
